(19) United States
(12) Patent Application Publication
(10) Pub. No.: US 2022/0028494 A1
(43) Pub. Date: Jan. 27, 2022

(54) METHODS AND SYSTEMS FOR DETERMINING THE CELLULAR ORIGIN OF CELL-FREE DNA

(71) Applicant: GUARDANT HEALTH, INC., Redwood City, CA (US)

(72) Inventors: OSCAR WESTESSON, Berkeley, CA (US); ELENA ZOTENKO, Sydney (AU)

(21) Appl. No.: 17/407,000

(22) Filed: Aug. 19, 2021

Related U.S. Application Data
(63) Continuation of application No. PCT/US20/19957, filed on Feb. 26, 2020.
(60) Provisional application No. 62/811,406, filed on Feb. 27, 2019.
(60) Provisional application No. 62/825,723, filed on Mar. 28, 2019.

(51) Int. Cl. G16B 35/20; G16B 30/10; G16B 40/00; G06N 7/00

(52) U.S. Cl. CPC G16B 35/20; G06N 7/005; G16B 40/00; G16B 30/10

(57) ABSTRACT

Provided herein are methods for determining the cellular origin of cell-free DNA. In one aspect, the methods include constructing a distribution of sequence and/or epigenetic information from DNA molecules obtained from a cfDNA sample over a plurality of base positions of a set of differential genomic sections or loci that comprise genomic regions and/or epigenetic loci. The differential genomic loci exhibit one or more properties that differ between at least two cell types. The methods also include processing the distribution of the sequence and/or epigenetic information from the DNA molecules over the set of the differential genomic loci to determine the cellular origin of at least a subset of DNA molecules from the cfDNA sample. Other aspects are directed to methods of treating disease in subjects. Yet other aspects include related systems and computer readable media used to determine the cellular origin of cfDNA.

### CROSS-REFERENCE TO RELATED APPLICATIONS

This application is a continuation of International Application No. PCT/US2020/019957, filed Feb. 26, 2020, which claims the benefit of, and relies on the filing dates of, U.S. provisional patent application no. 62/811,406, filed Feb. 27, 2019 and 62/825,723, filed Mar. 28, 2019, the entire disclosures of which are incorporated herein by reference.

### SEQUENCE LISTING

The instant application contains a Sequence Listing which has been submitted electronically in ASCII format and is hereby incorporated by reference in its entirety. Said ASCII copy, created on Feb. 25, 2020, is named GH0058WO_SL.txt and is 978 bytes in size.

### BACKGROUND

The early detection of cancer, for example, before tumors have metastasized or symptoms arise significantly improves associated mortality rates. Some methods of early stage cancer detection involve biomarker/genetic testing and liquid biopsies. Liquid biopsy assays are noninvasive tests that seek to detect tumor cells or cell-free DNA (cfDNA) originating from tumor cells (also known as, circulating tumor DNA (or ctDNA)) in blood samples taken from patients. cfDNA is often introduced into the bloodstream through apoptosis or necrosis, and typically have a half-life of only a few hours upon such introduction.

Currently, the most widely used liquid biopsy tests for cancer diagnosis, prognosis and clinical management generally rely on the presence of tumor originating somatic mutations in patient plasma cfDNA. In some situations, such as the analysis of cfDNA from patients with early stage cancer, the amount of tumor originating cfDNA in plasma is very small, which often makes detection of such mutations extremely challenging.

Thus, there remains a need to incorporate other signals present in plasma cfDNA samples to facilitate the detection of cancer as well as other diseases, disorders, or conditions.

### SUMMARY

This application discloses methods, computer readable media, and systems that are useful in determining the cellular origin of DNA molecules or cfDNA fragments from cfDNA samples, such as liquid biopsy samples. The methods disclosed herein facilitate the identification of the cellular source of nucleic acids, which are often present in very small quantities in cfDNA samples, such as in the case of tumor originating nucleic acids from early stage cancers. Accordingly, the methods and related aspects disclosed herein foster the early detection of disease, among numerous other applications.

In one aspect, this disclosure provides a method of determining a cellular origin of at least a subset of deoxyribonucleic acid (DNA) molecules (e.g., cfDNA fragments) from a cfDNA sample obtained from a subject at least partially using a computer. The method includes (a) identifying one or more sets of DNA molecules of unknown cellular origin from the cfDNA sample that each comprise one or more member DNA molecules that each comprise at least one genomic region in common with one another from sequence information obtained from the cfDNA sample. The method also includes (b) determining a distribution of one or more properties among the one or more member DNA molecules within each of the one or more sets of DNA molecules from epigenetic information and/or the sequence information obtained from the cfDNA sample to generate one or more distribution sets. The properties are selected from the group consisting of, for example, a length of a given DNA molecule, an offset of a midpoint of a given DNA molecule from a midpoint of the at least one genomic region of the given DNA molecule, an epigenetic status or pattern exhibited by a given DNA molecule, and/or the like. The method also includes (c) estimating a fraction of member DNA molecules, if any, within each of the one or more distribution sets that originate from a targeted cellular origin to generate a fraction estimate for each of the one or more distribution sets for the cfDNA sample. The method also includes (d) aggregating the fraction estimates for the cfDNA sample to generate a sample classification score for the cfDNA sample. In addition, the method also includes (e) classifying the cfDNA sample as comprising DNA molecules from cells of the targeted cellular origin when the sample classification score for the cfDNA sample exceeds a reference classification score, thereby determining the cellular origin of at least the subset of DNA molecules from the cfDNA sample obtained from the subject.

In one aspect, this disclosure provides a method of treating a disease in a subject. The method includes (a) identifying one or more sets of deoxyribonucleic acid (DNA) molecules of unknown cellular origin from a cell-free DNA (cfDNA) sample obtained from the subject that each comprise one or more member DNA molecules that each comprise at least one genomic region in common with one another from sequence information obtained from the cfDNA sample. The method also includes (b) determining a distribution of one or more properties among the one or more member DNA molecules within each of the one or more sets of DNA molecules from epigenetic information and/or the sequence information obtained from the cfDNA sample to generate one or more distribution sets, which properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The method also includes (c) estimating a fraction of member DNA molecules, if any, within each of the one or more distribution sets that originate from a targeted diseased cellular origin to generate a fraction estimate for each of the one or more distribution sets for the cfDNA sample. The method also includes (d) aggregating the fraction estimates for the cfDNA sample to generate a sample classification score for the cfDNA sample. The method also includes (e) classifying the cfDNA sample as comprising DNA molecules from cells of the targeted diseased cellular origin when the sample classification score for the cfDNA sample exceeds a reference classification score, thereby diagnosing the disease in the subject. In addition, the method also includes (f) administering one or more therapies to the subject based on the disease diagnosis, thereby treating the disease in the subject.

In one aspect, this disclosure provides a method of determining a cellular origin of at least a subset of deoxyribonucleic acid (DNA) molecules from a cell-free DNA (cfDNA) sample obtained from a subject (e.g., a mammalian subject, such as a human subject) at least partially using a computer. The method includes (a) determining, by the computer, a distribution of one or more properties within one or more sets of the DNA molecules from sequence and/or epigenetic information obtained from the DNA molecules in which each set of the DNA molecules comprises one or more members that each comprise one or more genomic regions in common with one another, and in which the one or more properties are selected from the group consisting of, for example, a length of a given DNA molecule (e.g., a number of nucleotides in the given DNA molecule), an offset of a midpoint of a given DNA molecule (e.g., a cfDNA fragment) from a midpoint of the one or more genomic regions of the given DNA molecule, an epigenetic status or pattern exhibited by a given DNA molecule, and/or the like. The method also includes (b) comparing, by the computer, the distribution of the one or more properties within the one or more sets of the DNA molecules, or a statistical transformation of one or more components of the distribution, to a reference distribution of the one or more properties within one or more sets of reference DNA molecules, or a statistical transformation of one or more components of the reference distribution. Each set of the reference DNA molecules comprises one or more members that each comprise one or more corresponding genomic regions in common with one another (e.g., corresponding to a genomic region in a set of DNA molecules from the cfDNA sample), which reference DNA molecules originate from one or more known cell types. A substantial match between the distribution of the one or more properties within the one or more sets of the DNA molecules, or the statistical transformation of the one or more components of the distribution, and the reference distribution of the one or more properties within the one or more sets of reference DNA molecules, or the statistical transformation of the one or more components of the reference distribution, indicates that at least the subset of the DNA molecules from the cfDNA sample originates from the one or more known cell types, thereby determining the cellular origin of at least the subset of the DNA molecules from the cfDNA sample obtained from the subject.

In another aspect, the disclosure provides a method of determining a cellular origin of at least a subset of deoxyribonucleic acid (DNA) molecules from a cell-free DNA (cfDNA) sample from a subject (e.g., a mammalian subject, such as a human subject) at least partially using a computer. The method includes (a) constructing, by the computer, at least one distribution of one or more properties obtained from the DNA molecules from the cfDNA sample, wherein the set of DNA molecules comprises member DNA molecules comprising one or more genomic regions and/or one or more epigenetic loci in common with one another, and wherein the one or more properties differ between at least two cell types. The method also includes (b) processing, by the computer, the distribution of the properties obtained from the DNA molecules to determine the cellular origin of at least the subset of DNA molecules from the cfDNA sample.

In another aspect, the disclosure provides a method treating a disease in a subject (e.g., a mammalian subject, such as a human subject). The method includes (a) determining a distribution of one or more properties within one or more sets of deoxyribonucleic acid (DNA) molecules obtained from a cell-free DNA (cfDNA) sample obtained from a subject from sequence and/or epigenetic information obtained from the DNA molecules. Each set of the DNA molecules comprises one or more members that each comprise one or more genomic regions in common with one another. The one or more properties are typically selected from the group consisting of, for example, a length of a given DNA molecule, an offset of a midpoint of a given DNA molecule from a midpoint of the one or more genomic regions of the given DNA molecule, an epigenetic status or pattern exhibited by a given DNA molecule, and/or the like. The method also includes (b) comparing the distribution of the one or more properties within the one or more sets of the DNA molecules, or a statistical transformation of one or more components of the distribution, to a reference distribution of the one or more properties within one or more sets of reference DNA molecules, or a statistical transformation of one or more components of the reference distribution. Each set of the reference DNA molecules comprises one or more members that each comprise one or more corresponding genomic regions in common with one another, which reference DNA molecules originate from one or more diseased cells. A substantial match between the distribution of the one or more properties within the one or more sets of the DNA molecules, or the statistical transformation of the one or more components of the distribution, and the reference distribution of the one or more properties within the one or more sets of reference DNA molecules, or the statistical transformation of the one or more components of the reference distribution, indicates that at least the subset of the DNA molecules from the cfDNA sample originates from the one or more diseased cells, thereby diagnosing the disease in the subject. In addition, the method also includes (c) administering one or more therapies to the subject based on the disease diagnosis, thereby treating the disease in the subject.

In some embodiments of the methods disclosed herein, the genomic regions comprise one or more regions of differential chromatin organization between at least two cell types. In certain embodiments, the genomic regions comprise, for example, one or more transcriptional factor binding regions (e.g., one or more CTCF binding regions), one or more distal regulatory elements (DREs), one or more repetitive elements, one or more intron-exon junctions, and/or one or more transcriptional start sites (TSSs).

In certain embodiments of the methods disclosed herein, the epigenetic loci comprise, for example, one or more methylation sites, one or more acetylation sites, one or more ubiquitylation sites, one or more phosphorylation sites, one or more sumoylation sites, one or more ribosylation sites, one or more citrullination sites, one or more histone post-translational modification sites, and/or one or more histone variant sites. In some embodiments, the epigenetic information comprises a methylation status of the one or more methylation sites, an acetylation status the one or more acetylation sites, a ubiquitylation status of the one or more ubiquitylation sites, a phosphorylation status of the one or more phosphorylation sites, a sumoylation status of the one or more sumoylation sites, a ribosylation status of the one or more ribosylation sites, a citrullination status of the one or more citrullination sites, a histone post-translational modification status of the one or more histone post-translational modification sites, a histone variant status of the one or more histone variant sites, and/or the like. Optionally, the epigenetic pattern comprises one or more of: a methylation pattern, an acetylation pattern, a ubiquitylation pattern, a phosphorylation pattern, a sumoylation pattern, a ribosylation pattern, a citrullination pattern, a histone post-translational modification pattern, and/or a histone variant pattern. In some of these embodiments, the methylation pattern comprises a 5-methylcytosine (5mC) pattern and/or a 5-hydroxymethylcytosine (5hmC) pattern.

The methods disclosed herein determine the cellular origin of essentially any cell type. In some embodiments, for example, the cellular origin of the subset of DNA molecules or the targeted cellular origin comprises a tumor cell. In certain embodiments, the cellular origin of the subset of DNA molecules or the targeted cellular origin comprises a non-tumor cell. In some embodiments, the cellular origin of the subset of DNA molecules or the targeted cellular origin comprises a fetal cell. In certain embodiments, the cellular origin of the subset of DNA molecules or the targeted cellular origin comprises a maternal cell. In certain embodiments, the cellular origin of the subset of DNA molecules or the targeted cellular origin comprises a cell from a transplant donor subject. In some embodiments, the cellular origin of the subset of DNA molecules or the targeted cellular origin comprises a cell from a transplant recipient subject. In certain embodiments, the cellular origin of the subset of DNA molecules or the targeted cellular origin comprises a non-diseased cell.

In some embodiments of the methods disclosed herein, the cellular origin of the subset of DNA molecules comprises a diseased cell, thereby diagnosing a disease in the subject. In certain of these embodiments, the methods further comprise administering one or more therapies to the subject to treat the disease in the subject. In some embodiments, the disease comprises cancer and wherein the therapies comprise at least one immunotherapy. In some of these embodiments, the immunotherapy comprises at least one checkpoint inhibitor antibody. In certain embodiments, the immunotherapy comprises an antibody against PD-1, PD-2, PD-L1, PD-L2, CTLA-40, OX40, B7.1, B7He, LAG3, CD137, KIR, CCR5, CD27, or CD40. In some embodiments, the immunotherapy comprises administration of a pro-inflammatory cytokine against at least one tumor type. In certain embodiments, the immunotherapy comprises administration of T cells against at least one tumor type.

The distribution of properties within sets of DNA molecules obtained from cfDNA samples determined according to the methods disclosed herein include various embodiments. In certain embodiments, for example, the one or more properties are selected from the group consisting of: a length of a given DNA molecule, an offset of a midpoint of a given DNA molecule from a midpoint of the one or more genomic regions of the given DNA molecule, an epigenetic status or pattern exhibited by a given DNA molecule, and/or the like. In some embodiments, the distribution comprises quantitative measures indicative of one or more of: (i) a number of the DNA molecules having a start point, a mid-point, or an end-point at each of the plurality of base positions; (ii) a length of the DNA molecules that align with each of the plurality of base positions; and, (iii) a number of the DNA molecules that align with each of the plurality of base positions.

In certain embodiments, the methods disclosed herein include applying one or more mixture models to generate the distribution estimate for each of the one or more differential genomic sections or to generate the fraction estimate for each of the one or more distribution sets for the cfDNA sample. In some embodiments, the methods include estimating a maximum likelihood that a fraction of DNA molecules in a given distribution set originates from the targeted cellular origin, using the equations of:

$${θ}_{ML} = arg {max}_{θ} Pr ( D \vert θ , Θ )$$

$$Pr ( D \vert θ , Θ ) = {Π}_{n} [ Pr ( {d}_{n} \vert {z}_{n} = targeted cell , Θ ) θ + Pr ( {d}_{n} \vert {z}_{n} = normal cell , Θ ) ( 1 - θ ) ]$$

where Pr is probability, θ is the fraction of DNA molecules in the given distribution set that originate from the targeted cellular origin, ML is the maximum likelihood, D is a collection of DNA molecules {d₁, d₂, . . . , d_{N}} from the test sample, n is a given DNA molecule in the given distribution set, d_{b} is a set of observed variables that represent observed fragmentomics and epigenetic information, zₙ is a latent/hidden variable that represents a targeted or normal cell of origin, and Θ is a set of parameters that are estimated from control genomic regions on a targeted panel or from a reference set of cfDNA samples with DNA molecules from normal cells and cfDNA samples with DNA molecules from targeted cells. In some of these embodiments, dₙ=(xₙ,yₙ,kₙ,qₙ), where n is the given DNA molecule in the given distribution set, xₙ is an offset of a midpoint of the given DNA molecule from a center of the genomic region of that given DNA molecule, yₙ is a length of the given DNA molecule, kₙ is a number of CpG sites in the given DNA molecule, and qₙ is a methyl binding domain (MBD) partition of the given DNA molecule.

In certain embodiments, the methods include generating the distribution estimate for each of the one or more differential genomic sections, or generating the fraction estimate for each of the one or more distribution sets for the cfDNA sample, using the equation of:

$$\begin{matrix}Pr ( x , y ) = & Pr ( x , y \vert active ) Pr ( active ) + \\ & Pr ( x , y \vert inactive ) Pr ( inactive ) \\ = & F ( x , y ) ( 1 - θ ) + H ( x , y ) θ\end{matrix}$$

where Pr is probability, x is offset of a midpoint of a given DNA molecule comprising a given genomic region with respect to a midpoint of the given genomic region, y is a nucleotide length of the given DNA molecule, θ is a fraction of DNA molecules originating from an inactive or diseased cellular source, F(x,y) is a density function for DNA molecules originating from an active or non-diseased cellular source and is estimated per given genomic region from the active or non-diseased cellular source in a train dataset, and H(x,y) is a density function for DNA molecules originating from an inactive or diseased cellular source and is estimated per sample. In some embodiments, the methods include using at least one maximum likelihood approach to estimate θ_{i,j} per region i and per sample j given F(x,y) and H(x,y). In some embodiments, the methods include generating the distribution estimate for each of the one or more differential genomic sections, or generating the fraction estimate for each of the one or more distribution sets for the cfDNA sample, using the equation of:

$$\begin{matrix}Pr ( x , y , z ) = & Pr ( x , y \vert z , active ) Pr ( z \vert active ) Pr ( active ) + \\ & Pr ( x , y \vert z , inactive ) Pr ( z \vert inactive ) Pr ( inactive ) \\ = & {F}_{z} ( x , y ) Pr ( z \vert active ) ( 1 - θ ) + \\ & {H}_{z} ( x , y ) Pr ( z \vert inactive ) θ\end{matrix}$$

where Pr is probability, x is offset of a midpoint of a given DNA molecule comprising a given genomic region with respect to a midpoint of the given genomic region, y is a nucleotide length of the given DNA molecule, z is an epigenetic state of the given DNA molecule, θ is a fraction of DNA molecules originating from an inactive or diseased cellular source, F_{z}(x,y) is a density function for DNA molecules originating from an active or non-diseased cellular source, H_{z}(x,y) is a density function for DNA molecules originating from an inactive or diseased cellular source. In certain embodiments, the methods include calculating an estimate of θ_{i,j} per region i and per sample j using the likelihood function of:

$$Pr ( D \vert θ ) = {Σ}_{β} Pr ( β ) Pr ( D \vert θ , α , β )$$

$$\begin{matrix}Pr ( D \vert θ , α , β ) = {Π}_{x , y , z = meth} ( 1 - θ ) {F}_{m} ( x , y ) α + θ {H}_{m} ( x , y ) β \\ {Π}_{x , y , z = unmeth} ( 1 - θ ) {F}_{u} ( x , y ) ( 1 - α ) + \\ θ {H}_{u} ( x , y ) ( 1 - β )\end{matrix}$$

where D is a collection of DNA molecules {d₁, d₂, . . . , d_{N}} from the test sample, Fₘ(x,y) is a density function for DNA molecules in a first epigenetic state and originating from an active or non-diseased cellular source and is estimated per given genomic region from the active or non-diseased cellular source in a train dataset, Fᵤ(x,y) is a density function for DNA molecules in a second epigenetic state and originating from an active or non-diseased cellular source and is estimated per given genomic region from the active or non-diseased cellular source in a train dataset, Hₘ(x,y) is a density function for DNA molecules in a first epigenetic state and originating from an inactive or diseased cellular source, Hᵤ(x,y) is a density function for DNA molecules in a first epigenetic state and originating from an active or non-diseased cellular source, α=Pr(z=first epigenetic state|active) is a fraction of DNA molecules in a first epigenetic state and originating from an active or non-diseased cellular source and is estimated per given genomic region from the active or non-diseased cellular source in a train dataset, and β=Pr(z=first epigenetic state|inactive) is a fraction of DNA molecules in a first epigenetic state and originating from an inactive or diseased cellular source. In some embodiments, the first epigenetic state comprises a methylated state and wherein the second epigenetic state comprises an unmethylated state. In some embodiments, a given DNA molecule is in a methylated state when the given DNA molecule is from a hyper or residual partition. In some embodiments, the methods include using cfDNA samples with DNA molecules originating from an active or non-diseased cellular source in the train dataset to estimate per given genomic region distributions of the θ values mean, μ_{θ}, and standard deviation, σ_{θ}. In some embodiments, the methods include transforming the 0 values to z-scores using the equation

$$\frac{θ - μ}{σ} .$$

In some embodiments, the methods include aggregating the z-scores for multiple genomic regions in a given cfDNA sample to generate a mean z-score to use as a classifier.

In some embodiments, the methods disclosed herein further include determining, by the computer, the presence or absence of one or more genetic aberrations in the subset of DNA molecules from the cfDNA sample. In some of these embodiments, the one or more genetic aberrations comprise one or more somatic mutations and/or germline mutations. In certain embodiments, the methods further comprise processing, by the computer, the distribution to determine a distribution score, wherein the distribution score is indicative of a mutation burden of the genetic aberration. Typically, processing, by the computer, comprises processing the distribution with one or more reference distributions obtained from cell-free DNA samples derived from one or more control subjects to determine the distribution score, wherein the distribution score indicates a difference between the distribution and the one or more reference distributions.

In some embodiments, the methods disclosed herein further include receiving the sequence and/or epigenetic information generated from the cfDNA sample. In certain embodiments, the methods further comprise receiving the sequence and the epigenetic information generated from the cfDNA sample. In some embodiments, the methods disclosed herein further include obtaining the cfDNA sample from the subject. Typically, the cfDNA sample is selected from the group consisting of, for example, tissue, blood, plasma, serum, sputum, urine, semen, vaginal fluid, feces, synovial fluid, spinal fluid, and saliva. In some embodiments, the methods disclosed herein further include generating the sequence and/or epigenetic information from the DNA molecules from the cfDNA sample. In some of these embodiments, the methods include amplifying one or more segments of the DNA molecules from the cfDNA sample to generate at least one amplified nucleic acid. In these embodiments, the methods typically further include sequencing the DNA molecules from the cfDNA sample to generate the sequence and/or epigenetic information. Optionally, the sequence and/or epigenetic information is obtained from targeted segments of nucleic acids in the cfDNA sample in which the targeted segments are obtained by selectively enriching one or more regions from the nucleic acids in the cfDNA sample prior to sequencing. In some embodiments, the methods further include amplifying the obtained targeted segments prior to sequencing. In certain embodiments, the methods further include attaching one or more adapters comprising barcodes to the nucleic acids prior to sequencing. Optionally, the sequencing is selected from the group consisting of, for example, targeted sequencing, bisulfite sequencing, intron sequencing, exome sequencing, whole genome sequencing, and/or the like.

In another aspect, the disclosure provides a method of generating a trained classifier using a computer. The method includes (a) identifying, by the computer, at least one set of one or more differential genomic sections that comprises one or more genomic regions and/or one or more epigenetic loci from sequence and/or epigenetic information from DNA molecules of a plurality of control samples of cell-free DNA (cfDNA). The method also includes (b) estimating, by the computer, a distribution of cfDNA of a given cellular origin for each of the one or more differential genomic sections identified from the control samples to generate a distribution estimate for each of the one or more differential genomic sections. In addition, the method also includes (c) aggregating, by the computer, the distribution estimates to generate a classifier score, thereby generating the trained classifier. In some of these embodiments, the given cellular origin of the cfDNA is tumor origin.

In some embodiments, the methods disclosed herein include identifying, by the computer, a cellular origin of one or more DNA molecules of cfDNA from a test sample from a subject using the trained classifier. In some embodiments, the methods include applying one or more mixture models to generate the distribution estimate for each of the one or more differential genomic sections. In certain embodiments, the methods include generating the distribution estimate for each of the one or more differential genomic sections using the equation of:

$$\begin{matrix}Pr ( x , y ) = & Pr ( x , y \vert active ) Pr ( active ) + \\ & Pr ( x , y \vert inactive ) Pr ( inactive ) \\ = & F ( x , y ) ( 1 - θ ) + H ( x , y ) θ\end{matrix}$$

where Pr is probability, x is offset of a midpoint of a given DNA molecule comprising a given genomic region with respect to a midpoint of the given genomic region, y is a nucleotide length of the given DNA molecule, θ is a fraction of DNA molecules originating from an inactive or diseased cellular source, F(x,y) is a density function for DNA molecules originating from an active or non-diseased cellular source and is estimated per given genomic region from the active or non-diseased cellular source in a train dataset, and H(x,y) is a density function for DNA molecules originating from an inactive or diseased cellular source and is estimated per sample. In some of these embodiments, the methods include using at least one maximum likelihood approach to estimate θ_{i,j} per region i and per sample j given F(x,y) and H(x,y).

In other exemplary embodiments, the methods disclosed herein include generating the distribution estimate for each of the one or more differential genomic sections using the equation of:

$$\begin{matrix}Pr ( x , y , z ) = & Pr ( x , y \vert z , active ) Pr ( z \vert active ) Pr ( active ) + \\ & Pr ( x , y \vert z , inactive ) Pr ( z \vert inactive ) Pr ( inactive ) \\ = & {F}_{z} ( x , y ) Pr ( z \vert active ) ( 1 - θ ) + \\ & {H}_{z} ( x , y ) Pr ( z \vert inactive ) θ\end{matrix}$$

where Pr is probability, x is offset of a midpoint of a given DNA molecule comprising a given genomic region with respect to a midpoint of the given genomic region, y is a nucleotide length of the given DNA molecule, z is an epigenetic state of the given DNA molecule, θ is a fraction of DNA molecules originating from an inactive or diseased cellular source, F_{z}(x,y) is a density function for DNA molecules originating from an active or non-diseased cellular source, H_{z}(x,y) is a density function for DNA molecules originating from an inactive or diseased cellular source. In certain embodiments, the methods include calculating an estimate of θ_{i,j} per region i and per sample j using the likelihood function of:

$$Pr ( D \vert θ ) = {Σ}_{β} Pr ( β ) Pr ( D \vert θ , α , β )$$

$$\begin{matrix}Pr ( D \vert θ , α , β ) = {Π}_{x , y , z = meth} ( 1 - θ ) {F}_{m} ( x , y ) α + θ {H}_{m} ( x , y ) β \\ {Π}_{x , y , z = unmeth} ( 1 - θ ) {F}_{u} ( x , y ) ( 1 - α ) + \\ θ {H}_{u} ( x , y ) ( 1 - β )\end{matrix}$$

where D is a collection of DNA molecules {d₁, d₂, . . . , d_{N}} from the test sample, Fₘ(x,y) is a density function for DNA molecules in a first epigenetic state and originating from an active or non-diseased cellular source and is estimated per given genomic region from the active or non-diseased cellular source in a train dataset, Fᵤ(x,y) is a density function for DNA molecules in a second epigenetic state and originating from an active or non-diseased cellular source and is estimated per given genomic region from the active or non-diseased cellular source in a train dataset, Hₘ(x,y) is a density function for DNA molecules in a first epigenetic state and originating from an inactive or diseased cellular source, Hᵤ(x,y) is a density function for DNA molecules in a first epigenetic state and originating from an active or non-diseased cellular source, α=Pr(z=first epigenetic state|active) is a fraction of DNA molecules in a first epigenetic state and originating from an active or non-diseased cellular source and is estimated per given genomic region from the active or non-diseased cellular source in a train dataset, and l=Pr(z=first epigenetic state|inactive) is a fraction of DNA molecules in a first epigenetic state and originating from an inactive or diseased cellular source. In some of these embodiments, the first epigenetic state comprises a methylated state and the second epigenetic state comprises an unmethylated state. In certain embodiments, a given DNA molecule is in a methylated state when the given DNA molecule is from a hyper or residual partition. In some embodiments, the methods disclosed herein include using samples with DNA molecules originating from an active or non-diseased cellular source in the train dataset to estimate per given genomic region distributions of the θ values mean, μ_{θ} and standard deviation, σ_{θ}. In some of these embodiments, the methods include transforming the θ values to z-scores using the equation

$$\frac{θ - μ}{σ} .$$

The methods also typically include aggregating the z-scores for multiple genomic regions in a given cfDNA sample to generate a mean z-score to use as the classifier.

In another aspect, the disclosure provides a method of classifying a test population of cell-free DNA (cfDNA) from a subject at least partially using a computer. The method includes (a) constructing, by the computer, a distribution of sequence and/or epigenetic information from the DNA molecules of the test population of cfDNA over a plurality of base positions of at least one set of one or more differential genomic sections that comprises one or more genomic regions and/or one or more epigenetic loci. The method also includes (b) processing, by the computer, the distribution of the sequence and/or epigenetic information from the DNA molecules using a trained classifier to classify the test population of cfDNA into one or more of a plurality of different classes corresponding to the distribution over the at least one set of one or more differential genomic sections that comprises the one or more genomic regions and the one or more epigenetic loci.

In another aspect, the disclosure provides a method of generating a trained classifier at least partially using a computer. The method includes (a) providing, by the computer, a plurality of different classes, wherein each class represents a set of subjects with a shared characteristic. The method also includes (b) for each of a plurality of populations of cell-free DNA (cfDNA) obtained from each of the classes, providing, by the computer, a distribution of DNA molecules of the population of cfDNA over a plurality of base positions of at least one set of one or more differential genomic sections that comprises one or more genomic regions and/or one or more epigenetic loci, and wherein the distribution of DNA molecules corresponds to a class of the classes, thereby providing a training data set. In addition, the method also includes (c) training a machine learning algorithm on the training data set to create one or more trained classifiers, wherein each trained classifier is configured to classify a test population of cfDNA from a test subject into one or more of the plurality of different classes.

In another aspect, the disclosure provides a method of identifying one or more biomarkers to use in determining a cellular origin of at least a subset of deoxyribonucleic acid (DNA) molecules from cell-free DNA (cfDNA) samples obtained from subjects at least partially using a computer. The method includes (a) identifying one or more sets of DNA molecules of a first known cellular origin from one or more first reference cfDNA samples, which sets of DNA molecules each comprise one or more member DNA molecules that each comprise at least one genomic region in common with one another from sequence information obtained from the first reference cfDNA samples. The method also includes (b) determining a distribution of one or more properties among the one or more member DNA molecules within each of the one or more sets of DNA molecules from epigenetic information and/or the sequence information obtained from the first reference cfDNA samples to generate one or more first distribution sets. The properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The method also includes (c) identifying one or more sets of DNA molecules of a second known cellular origin from one or more second reference cfDNA samples that each comprise one or more member DNA molecules that each comprise at least one corresponding genomic region in common with one another from sequence information obtained from the second reference cfDNA samples. The method also includes (d) determining a distribution of the properties among the one or more member DNA molecules within each of the one or more sets of DNA molecules from epigenetic information and/or the sequence information obtained from the second reference cfDNA samples to generate one or more second distribution sets. In addition, the method also includes (e) identifying one or more of the first and second distribution sets that comprise member DNA molecules that each comprise a given genomic region in common with one another and that comprise different distributions of the properties, thereby identifying the one or more biomarkers to use in determining the cellular origin of at least the subset of DNA molecules from cfDNA samples obtained from subjects.

In some embodiments, the first known cellular origin comprises non-diseased cells and wherein the second known cellular origin comprises diseased cells. In certain embodiments, the first known cellular origin comprises non-tumor cells and wherein the second known cellular origin comprises tumor cells. In some embodiments, the first known cellular origin comprises maternal cells and wherein the second known cellular origin comprises fetal cells. In some embodiments, the first known cellular origin comprises transplant recipient cells and wherein the second known cellular origin comprises transplant donor cells. In some embodiments, the genomic regions comprise one or more regions of differential chromatin organization between at least two cell types. In some embodiments, the genomic regions comprise one or more transcriptional factor binding regions (e.g., one or more CTCF binding regions), one or more distal regulatory elements (DREs), one or more repetitive elements, one or more intron-exon junctions, and/or one or more transcriptional start sites (TSSs).

In one aspect, this disclosure provides a method of generating a trained classifier at least partially using a computer. The method (a) identifying one or more sets of DNA molecules that each comprise one or more member DNA molecules that each comprise at least one genomic region in common with one another from sequence information obtained from at least one reference cell-free DNA (cfDNA) sample. The method also includes (b) determining a distribution of one or more properties among the one or more member DNA molecules within each of the one or more sets of DNA molecules from epigenetic information and/or the sequence information obtained from the reference cfDNA sample to generate one or more distribution sets. The properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The method also includes (c) estimating a fraction of member DNA molecules, if any, within each of the one or more distribution sets that originate from a targeted cellular origin to generate a fraction estimate for each of the one or more distribution sets for the reference cfDNA sample. In addition, the method includes (d) aggregating the fraction estimates for the reference cfDNA sample to generate a reference classification score, thereby generating the trained classifier. In some embodiments, the method further includes (e) generating a sample classification score for a test cfDNA sample obtained from a subject, and (f) classifying the test cfDNA sample as comprising DNA molecules from cells of the targeted cellular origin when the sample classification score for the test cfDNA sample exceeds the reference classification score, thereby determining the cellular origin of at least a subset of DNA molecules from the test cfDNA sample obtained from the subject.

In another aspect, the disclosure provides a system, comprising a controller comprising, or capable of accessing, computer readable media comprising non-transitory computer-executable instructions which, when executed by at least one electronic processor perform at least: (a) receiving sequence and/or epigenetic information obtained from DNA molecules from a cell-free DNA (cfDNA) sample, (b) constructing at least one distribution of one or more properties obtained from the sequence and/or epigenetic information from at least one set of the DNA molecules, wherein the set of DNA molecules comprises member DNA molecules comprising one or more genomic regions and/or one or more epigenetic loci in common with one another, and wherein the one or more properties differ between at least two cell types, and (c) processing the distribution of the properties obtained from the sequence and/or epigenetic information to determine a cellular origin of at least a subset of DNA molecules from the cfDNA sample.

In yet another aspect, the disclosure provides a system, comprising a controller comprising, or capable of accessing, computer readable media comprising non-transitory computer-executable instructions which, when executed by at least one electronic processor perform at least: (a) receiving sequence and/or epigenetic information obtained from DNA molecules in a cell-free DNA (cfDNA) sample obtained from a subject. The computer readable media also include non-transitory computer-executable instructions which, when executed by at least one electronic processor perform at least: (b) determining a distribution of one or more properties within one or more sets of the DNA molecules from the sequence and/or epigenetic information, wherein each set of the DNA molecules comprises one or more members that each comprise one or more genomic regions in common with one another, and wherein the one or more properties are selected from the group consisting of: a length of a given DNA molecule, an offset of a midpoint of a given DNA molecule from a midpoint of the one or more genomic regions of the given DNA molecule, and an epigenetic status or pattern exhibited by a given DNA molecule. In addition, the computer readable media also include non-transitory computer-executable instructions which, when executed by at least one electronic processor perform at least: (c) comparing the distribution of the one or more properties within the one or more sets of the DNA molecules, or a statistical transformation of one or more components of the distribution, to a reference distribution of the one or more properties within one or more sets of reference DNA molecules, or a statistical transformation of one or more components of the reference distribution, wherein each set of the reference DNA molecules comprises one or more members that each comprise one or more corresponding genomic regions in common with one another, which reference DNA molecules originate from one or more known cell types, wherein a substantial match between the distribution of the one or more properties within the one or more sets of the DNA molecules, or the statistical transformation of the one or more components of the distribution, and the reference distribution of the one or more properties within the one or more sets of reference DNA molecules, or the statistical transformation of the one or more components of the reference distribution, indicates that at least a subset of the DNA molecules from the cfDNA sample originates from the one or more known cell types, thereby determining the cellular origin of at least the subset of the DNA molecules from the cfDNA sample obtained from the subject.

In another aspect, the disclosure provides a system, comprising a controller comprising, or capable of accessing, computer readable media comprising non-transitory computer-executable instructions which, when executed by at least one electronic processor perform at least: (a) identifying one or more sets of deoxyribonucleic acid (DNA) molecules of unknown cellular origin from the cell-free DNA (cfDNA) sample that each comprise one or more member DNA molecules that each comprise at least one genomic region in common with one another from sequence information obtained from the cfDNA sample. The system also includes (b) determining a distribution of one or more properties among the one or more member DNA molecules within each of the one or more sets of DNA molecules from epigenetic information and/or the sequence information obtained from the cfDNA sample to generate one or more distribution sets. The properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The system also includes (c) estimating a fraction of member DNA molecules, if any, within each of the one or more distribution sets that originate from a targeted cellular origin to generate a fraction estimate for each of the one or more distribution sets for the cfDNA sample. The system also includes (d) aggregating the fraction estimates for the cfDNA sample to generate a sample classification score for the cfDNA sample. In addition, the system also includes (e) classifying the cfDNA sample as comprising DNA molecules from cells of the targeted cellular origin when the sample classification score for the cfDNA sample exceeds a reference classification score.

In another aspect, the disclosure provides a system, comprising a controller comprising, or capable of accessing, computer readable media comprising non-transitory computer-executable instructions which, when executed by at least one electronic processor perform at least: (a) identifying one or more sets of deoxyribonucleic acid (DNA) molecules of a first known cellular origin from one or more first reference cell-free DNA (cfDNA) samples. The sets of DNA molecules each comprise one or more member DNA molecules that each comprise at least one genomic region in common with one another from sequence information obtained from the first reference cfDNA samples. The system also includes (b) determining a distribution of one or more properties among the one or more member DNA molecules within each of the one or more sets of DNA molecules from epigenetic information and/or the sequence information obtained from the first reference cfDNA samples to generate one or more first distribution sets. The properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The system also includes (c) identifying one or more sets of DNA molecules of a second known cellular origin from one or more second reference cfDNA samples that each comprise one or more member DNA molecules that each comprise at least one corresponding genomic region in common with one another from sequence information obtained from the second reference cfDNA samples. The system also includes (d) determining a distribution of the properties among the one or more member DNA molecules within each of the one or more sets of DNA molecules from epigenetic information and/or the sequence information obtained from the second reference cfDNA samples to generate one or more second distribution sets. In addition, the system also includes (e) identifying one or more of the first and second distribution sets that comprise member DNA molecules that each comprise a given genomic region in common with one another and that comprise different distributions of the properties, thereby identifying one or more biomarkers to use in determining a cellular origin of at least a subset of DNA molecules from cfDNA samples obtained from subjects.

In another aspect, the disclosure provides a system, comprising a controller comprising, or capable of accessing, computer readable media comprising non-transitory computer-executable instructions which, when executed by at least one electronic processor perform at least: (a) identifying one or more sets of DNA molecules that each comprise one or more member DNA molecules that each comprise at least one genomic region in common with one another from sequence information obtained from at least one reference cell-free DNA (cfDNA) sample. The system also includes (b) determining a distribution of one or more properties among the one or more member DNA molecules within each of the one or more sets of DNA molecules from epigenetic information and/or the sequence information obtained from the reference cfDNA sample to generate one or more distribution sets, which properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The system also includes (c) estimating a fraction of member DNA molecules, if any, within each of the one or more distribution sets that originate from a targeted cellular origin to generate a fraction estimate for each of the one or more distribution sets for the reference cfDNA sample, and (d) aggregating the fraction estimates for the reference cfDNA sample to generate a reference classification score.

In some embodiments, the systems disclosed herein include a nucleic acid sequencer operably connected to the controller, which nucleic acid sequencer is configured to provide the sequence and/or epigenetic information from the DNA molecules in the cfDNA sample. Typically, the nucleic acid sequencer is configured to perform, for example, pyrosequencing, bisulfite sequencing, single-molecule sequencing, nanopore sequencing, semiconductor sequencing, sequencing-by-ligation or sequencing-by-hybridization on the nucleic acids to generate sequencing reads. In some embodiments, the systems include a sample preparation component operably connected to the controller, which sample preparation component is configured to prepare the DNA molecules to be sequenced by a nucleic acid sequencer. In some of these embodiments, the sample preparation component is configured to selectively enrich regions from the DNA molecules in the cfDNA sample. In some embodiments, the sample preparation component is configured to attach one or adapters comprising barcodes to the DNA molecules. In certain embodiments, the systems disclosed herein include a nucleic acid amplification component operably connected to the controller, which nucleic acid amplification component is configured to amplify the DNA molecules. The nucleic acid amplification component is optionally configured to amplify selectively enriched regions from the DNA molecules in the cfDNA sample. In some embodiments, the systems disclosed herein include a material transfer component operably connected to the controller, which material transfer component is configured to transfer one or more materials between a nucleic acid sequencer and a sample preparation component. In certain embodiments, the systems disclosed herein include a database operably connected to the controller, which database comprises at least one reference distribution of the one or more properties within the one or more sets of reference DNA molecules, or the statistical transformation of the one or more components of the reference distribution.

In another aspect, the disclosure provides a computer readable media comprising non-transitory computer-executable instructions which, when executed by at least one electronic processor perform at least: (a) receiving sequence and/or epigenetic information obtained from DNA molecules in a cell-free DNA (cfDNA) sample, (b) constructing at least one distribution of one or more properties obtained from the sequence and/or epigenetic information from at least one set of the DNA molecules, wherein the set of DNA molecules comprises member DNA molecules comprising one or more genomic regions and/or one or more epigenetic loci in common with one another, and wherein the one or more properties differ between at least two cell types, and (c) processing the distribution of the properties obtained from the sequence and/or epigenetic information to determine a cellular origin of at least a subset of DNA molecules from the cfDNA sample.

In still another aspect, the disclosure provides a computer readable media comprising non-transitory computer-executable instructions which, when executed by at least one electronic processor perform at least: (a) receiving sequence and/or epigenetic information obtained from DNA molecules in a cell-free DNA (cfDNA) sample obtained from a subject. The computer readable media also includes non-transitory computer-executable instructions which, when executed by at least one electronic processor perform at least: (b) determining a distribution of one or more properties within one or more sets of the DNA molecules from the sequence and/or epigenetic information, wherein each set of the DNA molecules comprises one or more members that each comprise one or more genomic regions in common with one another, and wherein the one or more properties are selected from the group consisting of: a length of a given DNA molecule, an offset of a midpoint of a given DNA molecule from a midpoint of the one or more genomic regions of the given DNA molecule, and an epigenetic status or pattern exhibited by a given DNA molecule. In addition, the computer readable media also includes non-transitory computer-executable instructions which, when executed by at least one electronic processor perform at least: (c) comparing the distribution of the one or more properties within the one or more sets of the DNA molecules, or a statistical transformation of one or more components of the distribution, to a reference distribution of the one or more properties within one or more sets of reference DNA molecules, or a statistical transformation of one or more components of the reference distribution, wherein each set of the reference DNA molecules comprises one or more members that each comprise one or more corresponding genomic regions in common with one another, which reference DNA molecules originate from one or more known cell types, wherein a substantial match between the distribution of the one or more properties within the one or more sets of the DNA molecules, or the statistical transformation of the one or more components of the distribution, and the reference distribution of the one or more properties within the one or more sets of reference DNA molecules, or the statistical transformation of the one or more components of the reference distribution, indicates that at least a subset of the DNA molecules from the cfDNA sample originates from the one or more known cell types, thereby determining the cellular origin of at least the subset of the DNA molecules from the cfDNA sample obtained from the subject.

In another aspect, the disclosure provides a computer readable media comprising non-transitory computer-executable instructions which, when executed by at least one electronic processor perform at least: (a) identifying one or more sets of deoxyribonucleic acid (DNA) molecules of unknown cellular origin from the cell-free DNA (cfDNA) sample that each comprise one or more member DNA molecules that each comprise at least one genomic region in common with one another from sequence information obtained from the cfDNA sample. The computer readable media also includes (b) determining a distribution of one or more properties among the one or more member DNA molecules within each of the one or more sets of DNA molecules from epigenetic information and/or the sequence information obtained from the cfDNA sample to generate one or more distribution sets. The properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The computer readable media also includes (c) estimating a fraction of member DNA molecules, if any, within each of the one or more distribution sets that originate from a targeted cellular origin to generate a fraction estimate for each of the one or more distribution sets for the cfDNA sample. The computer readable media also includes (d) aggregating the fraction estimates for the cfDNA sample to generate a sample classification score for the cfDNA sample. In addition, the computer readable media also includes (e) classifying the cfDNA sample as comprising DNA molecules from cells of the targeted cellular origin when the sample classification score for the cfDNA sample exceeds a reference classification score.

In another aspect, the disclosure provides a computer readable media comprising non-transitory computer-executable instructions which, when executed by at least one electronic processor perform at least: (a) identifying one or more sets of deoxyribonucleic acid (DNA) molecules of a first known cellular origin from one or more first reference cell-free DNA (cfDNA) samples. The sets of DNA molecules each comprise one or more member DNA molecules that each comprise at least one genomic region in common with one another from sequence information obtained from the first reference cfDNA samples. The computer readable media also includes (b) determining a distribution of one or more properties among the one or more member DNA molecules within each of the one or more sets of DNA molecules from epigenetic information and/or the sequence information obtained from the first reference cfDNA samples to generate one or more first distribution sets. The properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The computer readable media also includes (c) identifying one or more sets of DNA molecules of a second known cellular origin from one or more second reference cfDNA samples that each comprise one or more member DNA molecules that each comprise at least one corresponding genomic region in common with one another from sequence information obtained from the second reference cfDNA samples. The computer readable media also includes (d) determining a distribution of the properties among the one or more member DNA molecules within each of the one or more sets of DNA molecules from epigenetic information and/or the sequence information obtained from the second reference cfDNA samples to generate one or more second distribution sets. In addition, the computer readable media also includes (e) identifying one or more of the first and second distribution sets that comprise member DNA molecules that each comprise a given genomic region in common with one another and that comprise different distributions of the properties, thereby identifying one or more biomarkers to use in determining a cellular origin of at least a subset of DNA molecules from cfDNA samples obtained from subjects.

In another aspect, the disclosure provides a computer readable media comprising non-transitory computer-executable instructions which, when executed by at least one electronic processor perform at least: (a) identifying one or more sets of DNA molecules that each comprise one or more member DNA molecules that each comprise at least one genomic region in common with one another from sequence information obtained from at least one reference cell-free DNA (cfDNA) sample. The system also includes (b) determining a distribution of one or more properties among the one or more member DNA molecules within each of the one or more sets of DNA molecules from epigenetic information and/or the sequence information obtained from the reference cfDNA sample to generate one or more distribution sets, which properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule. The system also includes (c) estimating a fraction of member DNA molecules, if any, within each of the one or more distribution sets that originate from a targeted cellular origin to generate a fraction estimate for each of the one or more distribution sets for the reference cfDNA sample, and (d) aggregating the fraction estimates for the reference cfDNA sample to generate a reference classification score.

In some embodiments of the systems or computer readable media disclosed herein, the computer readable media comprising non-transitory computer-executable instructions which, when executed by the at least one electronic processor further perform at least: applying one or more mixture models to generate the distribution estimate for each of the one or more differential genomic sections or to generate the fraction estimate for each of the one or more distribution sets for the cfDNA sample. In some embodiments of the systems or computer readable media disclosed herein, the computer readable media comprises non-transitory computer-executable instructions which, when executed by the at least one electronic processor further perform at least: estimating a maximum likelihood that a fraction of DNA molecules in a given distribution set originates from the targeted cellular origin, using the equations of:

$${θ}_{ML} = arg {max}_{θ} Pr ( D \vert θ , Θ )$$

$$Pr ( D \vert θ , Θ ) = {Π}_{n} [ Pr ( {d}_{n} \vert {z}_{n} = targeted cell , Θ ) θ + Pr ( {d}_{n} \vert {z}_{n} = normal cell , Θ ) ( 1 - θ ) ]$$

where Pr is probability, θ is the fraction of DNA molecules in the given distribution set that originate from the targeted cellular origin, ML is the maximum likelihood, D is a collection of DNA molecules {d₁, d₂, . . . , d_{N}} from the new sample, n is a given DNA molecule in the given distribution set, dₙ is a set of observed variables that represent observed fragmentomics and epigenetic information, zₙ is a latent/hidden variable that represents a targeted or normal cell of origin, and θ is a set of parameters that are estimated from control genomic regions on a targeted panel or from a reference set of cfDNA samples with DNA molecules from normal cells and cfDNA samples with DNA molecules from targeted cells. In certain of these embodiments, dₙ=(xₙ,yₙ,kₙ,qₙ), where n is the given DNA molecule in the given distribution set, xₙ is an offset of a midpoint of the given DNA molecule from a center of the genomic region of that given DNA molecule, yₙ, is a length of the given DNA molecule, kₙ is a number of CpG sites in the given DNA molecule, and qₙ is a methyl binding domain (MBD) partition of the given DNA molecule.

In some embodiments of the systems or computer readable media disclosed herein, the computer readable media comprising non-transitory computer-executable instructions which, when executed by the at least one electronic processor further perform at least: generating the distribution estimate for each of the one or more differential genomic sections, or generating the fraction estimate for each of the one or more distribution sets for the cfDNA sample, using the equation of:

$$\begin{matrix}Pr ( x , y ) = & Pr ( x , y \vert active ) + Pr ( active ) + \\ & Pr ( x , y \vert inactive ) Pr ( inactive ) \\ = & F ( x , y ) ( 1 - θ ) + H ( x , y ) θ\end{matrix}$$

where Pr is probability, x is offset of a midpoint of a given DNA molecule comprising a given genomic region with respect to a midpoint of the given genomic region, y is a nucleotide length of the given DNA molecule, θ is a fraction of DNA molecules originating from an inactive or diseased cellular source, F(x,y) is a density function for DNA molecules originating from an active or non-diseased cellular source and is estimated per given genomic region from the active or non-diseased cellular source in a train dataset, and H(x,y) is a density function for DNA molecules originating from an inactive or diseased cellular source and is estimated per sample. In some embodiments of the systems or computer readable media disclosed herein, the computer readable media comprising non-transitory computer-executable instructions which, when executed by the at least one electronic processor further perform at least: using at least one maximum likelihood approach to estimate Bi per region i and per sample j given F(x,y) and H(x,y). In some embodiments of the systems or computer readable media disclosed herein, the computer readable media comprising non-transitory computer-executable instructions which, when executed by the at least one electronic processor further perform at least: generating the distribution estimate for each of the one or more differential genomic sections, or generating the fraction estimate for each of the one or more distribution sets for the cfDNA sample, using the equation of:

$$\begin{matrix}Pr ( x , y , z ) = & Pr ( x , y \vert z , active ) Pr ( z \vert active ) Pr ( active ) + \\ & Pr ( x , y \vert z , inactive ) Pr ( z \vert inactive ) Pr ( inactive ) \\ = & {F}_{z} ( x , y ) Pr ( z \vert active ) ( 1 - θ ) + \\ & {H}_{z} ( x , y ) Pr ( z \vert inactive ) θ\end{matrix}$$

where Pr is probability, x is offset of a midpoint of a given DNA molecule comprising a given genomic region with respect to a midpoint of the given genomic region, y is a nucleotide length of the given DNA molecule, z is an epigenetic state of the given DNA molecule, θ is a fraction of DNA molecules originating from an inactive or diseased cellular source, F_{z}(x,y) is a density function for DNA molecules originating from an active or non-diseased cellular source, H_{z}(x,y) is a density function for DNA molecules originating from an inactive or diseased cellular source. In some embodiments of the systems or computer readable media disclosed herein, the computer readable media comprising non-transitory computer-executable instructions which, when executed by the at least one electronic processor further perform at least: calculating an estimate of Bi per region i and per sample j using the likelihood function of:

$$Pr ( D \vert θ ) = {Σ}_{β} Pr ( β ) Pr ( D \vert θ , α , β )$$

$$\begin{matrix}Pr ( D \vert θ , α , β ) = {Π}_{x , y , z = meth} ( 1 - θ ) {F}_{m} ( x , y ) α + θ {H}_{m} ( x , y ) β \\ {Π}_{x , y , z = unmeth} ( 1 - θ ) {F}_{u} ( x , y ) ( 1 - θ ) + \\ θ {H}_{u} ( x , y ) ( 1 - β )\end{matrix}$$

where D is a collection of DNA molecules {d₁, d₂, . . . , d_{N}} from the test sample, Fₘ(x,y) is a density function for DNA molecules in a first epigenetic state and originating from an active or non-diseased cellular source and is estimated per given genomic region from the active or non-diseased cellular source in a train dataset, Fᵤ(x,y) is a density function for DNA molecules in a second epigenetic state and originating from an active or non-diseased cellular source and is estimated per given genomic region from the active or non-diseased cellular source in a train dataset, Hₘ(x,y) is a density function for DNA molecules in a first epigenetic state and originating from an inactive or diseased cellular source, Hᵤ(x,y) is a density function for DNA molecules in a first epigenetic state and originating from an active or non-diseased cellular source, α=Pr(z=first epigenetic state|active) is a fraction of DNA molecules in a first epigenetic state and originating from an active or non-diseased cellular source and is estimated per given genomic region from the active or non-diseased cellular source in a train dataset, and l=Pr(z=first epigenetic state|inactive) is a fraction of DNA molecules in a first epigenetic state and originating from an inactive or diseased cellular source. In some embodiments of the systems or computer readable media disclosed herein, the computer readable media comprising non-transitory computer-executable instructions which, when executed by the at least one electronic processor further perform at least: using cfDNA samples with DNA molecules originating from an active or non-diseased cellular source in the train dataset to estimate per given genomic region distributions of the θ values mean, μ_{θ}, and standard deviation, σ_{θ}. In some embodiments of the systems or computer readable media disclosed herein, the computer readable media comprising non-transitory computer-executable instructions which, when executed by the at least one electronic processor further perform at least: transforming the θ values to z-scores using the equation

$$\frac{θ - μ}{σ} .$$

In some embodiments of the systems or computer readable media disclosed herein, the computer readable media comprising non-transitory computer-executable instructions which, when executed by the at least one electronic processor further perform at least: aggregating the z-scores for multiple genomic regions in a given cfDNA sample to generate a mean z-score to use as a classifier.

### BRIEF DESCRIPTION OF THE DRAWINGS

The accompanying drawings, which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps of determining the cellular origin of DNA molecules from a cfDNA sample according to some embodiments of the invention.

FIG. 2 is a flow chart that schematically depicts exemplary method steps of determining the cellular origin of DNA molecules from a cfDNA sample according to some embodiments of the invention.

FIG. 3 is a flow chart that schematically depicts exemplary method steps of classifying a test population of cfDNA molecules according to some embodiments of the invention.

FIG. 4 is a flow chart that schematically depicts exemplary method steps of generating a trained classifier according to some embodiments of the invention.

FIG. 5 is a flow chart that schematically depicts exemplary method steps of generating a trained classifier according to some embodiments of the invention.

FIG. 6 is a flow chart that schematically depicts exemplary method steps of identifying biomarkers to use in determining the cellular origin of DNA molecules from cfDNA samples according to some embodiments of the invention.

FIG. 7 is a schematic diagram of an exemplary system suitable for use with certain embodiments of the invention.

FIG. 8 shows a plot of a representative CTCF profile.

FIG. 9 shows a plot of a number of identified CTCF sites as a function of distance cut-off.

FIG. 10 shows a plot of a fraction of known sites identified as a function of distance cut-off.

FIG. 11 is a genome browser screenshot showing an example of an inferred CTCF site within an intronic region of the RBFOX1 gene. The genome browser tracks include GENCODE V18 and RefSeq gene annotations.

FIG. 12 is a genome browser screenshot of an inferred CTCF binding region—CTCF_INFRD_3375—mapping to the promoter of the THBD gene. The genome browser tracks include GENCODE V18 and RefSeq gene annotations, inferred CTCF region boundaries, panel probes covering the selected region, 25th and 75th DNA methylation level quantiles derived from public blood methylation data, 25th and 75th DNA methylation level quantiles derived from Cancer Genome Atlas Colon Adenocarcinoma (TCGA COAD) tumor and adjacent normal samples, 25th and 75th DNA methylation level quantiles derived from Cancer Genome Atlas Lung Adenocarcinoma (TCGA LUAD) tumor and adjacent normal samples.

FIG. 13 is a genome browser screenshot of an inferred CTCF binding region—CTCF_INFRD_20483—mapping to the promoter distal locus on chromosome 1. The genome browser tracks include GENCODE V18 and RefSeq gene annotations, inferred CTCF region boundaries, panel probes covering the selected region, 25th and 75th DNA methylation level quantiles derived from public blood methylation data, 25th and 75th DNA methylation level quantiles derived from TCGA COAD tumor and adjacent normal samples, 25th and 75th DNA methylation level quantiles derived from TCGA LUAD tumor and adjacent normal samples.

FIGS. 14A-D are plots of computed active and inactive densities for the CTCF_INFRD_3375 region. The color gradient encodes the probability values across offset values ranging from −200 bp to 200 bp on the x-axis and fragment length values ranging from 90 bp to 240 bp on the y-axis; offset values correspond to or are relative to the center or midpoint of the inferred CTCF binding site. More specifically, FIG. 14A is a plot of the active density computed from a set of train Normal cfDNA samples. FIG. 14B is a plot of the tumor density computed from a set of train Late Stage High-MAF Tumor cfDNA samples. FIG. 14C is a plot of the inactive density derived through a Maximum Likelihood Estimation process. FIG. 14D is a plot of the reconstructed tumor density using estimated active and inactive densities and fixed value for inactive component fraction θ=0.1.

FIGS. 15A-D are plots of computed active and inactive densities for the CTCF_INFRD_20483 region. The color gradient encodes the probability values across offset values ranging from −200 bp to 200 bp on the x-axis and fragment length values ranging from 90 bp to 240 bp on the y-axis; offset values correspond to the center of the inferred CTCF binding site. More specifically, FIG. 15A is a plot of the active density computed from a set of train Normal cfDNA samples. FIG. 15B is a plot of the tumor density computed from a set of train Late Stage High-MAF Tumor cfDNA samples. FIG. 15C is a plot of the inactive density derived through a Maximum Likelihood Estimation process. FIG. 15D is a plot of the reconstructed tumor density using estimated active and inactive densities and fixed value for inactive component fraction θ=0.1.

FIGS. 16 A and B are plots showing the performance of an exemplary fragmentomics only model on several cohorts of cfDNA samples. Model derived scores for cfDNA samples from two cohorts of Early Stage colorectal cancer (CRC) patients (n1=59 and n2=22) and one cohort of Late Stage CRC Low MAF patients (n=15) are compared to scores from cfDNA samples from a cohort of age matched healthy donors (n=70). In particular, FIG. 16A are ROC curves showing sensitivity and specificity of model derived mean z-score values. FIG. 16B is a scatter plot showing the distribution of model derived mean z-score values (meanZscore on the x-axis) and number of regions with z-score value above 3.0 (numLociPositive on the y-axis). Samples and ROC curves are color-coded by the cohort.

FIGS. 17 A and B are plots showing the performance of an exemplary fragmentomics and DNA methylation combined model on several cohorts of cfDNA samples. Model derived scores for cfDNA samples from two cohorts of Early Stage CRC patients (n1=59 and n2=22) and one cohort of Late Stage CRC Low MAF patients (n=15) are compared to scores from cfDNA samples from a cohort of age matched healthy donors (n=70). In particular, FIG. 17A are ROC curves showing sensitivity and specificity of model derived mean z-score values. FIG. 17B is a scatter plot showing the distribution of model derived mean z-score values (meanZscore on the x-axis) and number of regions with z-score value above 3.0 (numLociPositive on the y-axis). Samples and ROC curves are color-coded by the cohort.

### DEFINITIONS

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Active: As used herein, “active” in the context of cfDNA fragments or molecules refers to molecules that originate from normal or non-diseased cells (e.g., non-tumor cells). In certain embodiments, for example, a given “active” genomic region is a CTCF binding region that is bound by a CTCF transcription factor.

Adapter: As used herein, “adapter” refers to short nucleic acids (e.g., less than about 500, less than about 100 or less than about 50 nucleotides in length) that are typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequencing reads of a given nucleic acid molecule. The same or different adapters can be linked to the respective ends of a nucleic acid molecule. In certain embodiments, the same adapter is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides. In still other exemplary embodiments, an adapter is a bell-shaped adapter that includes a blunt or tailed end for joining to a nucleic acid molecule to be analyzed. Other exemplary adapters include T-tailed and C-tailed adapters.

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” in the context of nucleic acids refers to a nucleic acid molecule comprising a sequence that can serve as a molecular identifier. For example, individual “barcode” sequences are typically added to each DNA fragment during next-generation sequencing (NGS) library preparation so that each read can be identified and sorted before the final data analysis.

Cancer Type: As used herein, “cancer,” “cancer type” or “tumor type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell or, in some embodiments, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Cell-free nucleic acids can be found in an efferosome or an exosome. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Origin: As used herein, “cellular origin” in the context of cell-free nucleic acids means the cell type from which a given cell-free nucleic acid molecule derives or otherwise originates (e.g., via a apoptotic process, a necrotic process, or the like). In certain embodiments, for example, a given cell-free nucleic acid molecule may originate from a tumor cell (e.g., a cancerous pulmonary cell, etc.) or a non-tumor or normal cell (e.g., a non-cancerous pulmonary cell, etc.).

Comparator Result: As used herein, “comparator result” or “reference result” means a result or set of results to which a given test sample or test result can be compared to identify one or more likely properties of the test sample or result, and/or one or more possible prognostic outcomes and/or one or more customized therapies for the subject from whom the test sample was taken or otherwise derived. Comparator results are typically obtained from a set of reference samples (e.g., from subjects having the same disease or cancer type as the test subject and/or from subjects who are receiving, or who have received, the same therapy as the test subject).

Control Sample: As used herein, “control sample” or “control DNA sample” refers a sample of known composition and/or having known properties and/or known parameters (e.g., known cellular origin, known tumor fraction, known coverage, and/or the like) that is analyzed along with or compared to test samples in order to evaluate the accuracy of an analytical procedure. A control sample dataset typically includes from at least about 25 to at least about 30,000 or more control samples. In some embodiments, the control sample dataset includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more control samples.

Corresponding genetic region: As used herein, “corresponding genetic region” refers to a genetic region that two or more given DNA molecules comprise in common with one another. For example, a test cfDNA fragment and a control cfDNA fragment may include the same CTCF binding site in common with one another.

Coverage: As used herein, “coverage” refers to the number of nucleic acid molecules that represent a particular base position.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising four types of nucleotides; adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising four types of nucleotides; A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Differential Genomic Sections: As used herein, “differential genomic section” means a section of a given genome that comprises a given genomic region (e.g., a given transcription factor binding site or region, transcriptional start site (TSS), distal regulatory element (DRE), or the like) and which exhibits one or more different properties (e.g., variable chromatin organization patterns and variable epigenetic states) in at least two different cell or tissue types.

Epigenetic Information: As used herein, “epigenetic information” in the context of a DNA polymer means one or more epigenetic patterns exhibited in that polymer.

Epigenetic Locus: As used herein, “epigenetic locus” or “epigenetic site” means a fixed position on a chromosome that exhibits different states or statuses that do not involve changes or alterations in nucleotide sequence. For example, a given epigenetic locus may or may not be acetylated, methylated (e.g., modified with 5-methylcytosine (5mC), modified with 5-hydroxymethylcytosine (5hmC), and/or the like), ubiquitylated, phosphorylated, sumoylated, ribosylated, citrullinated, have a histone post-translational modification or other histone variation, and/or the like.

Epigenetic Pattern: As used herein, “epigenetic pattern” means an epigenetic state or status exhibited by one or more epigenetic loci in a given DNA molecule. For example, DNA molecules or cfDNA fragments that comprise a given genomic region or locus (e.g., a CTCF binding region, etc.) may also exhibit epigenetic patterns in which some of those DNA molecules include a certain number of epigenetic loci that are methylated, whereas in other instances corresponding epigenetic loci in other DNA molecules or cfDNA fragments that comprise the same genomic region are unmethylated.

Genomic Region: As used herein, “genomic region” means a fixed position on, or section of, a chromosome, such as the position of a gene or a genomic marker. Exemplary genomic markers include transcriptional factor binding regions (e.g., CTCF binding regions, etc.), distal regulatory elements (DREs), repetitive elements (e.g., microsatellites, etc.), intron-exon or exon-intron junctions, transcriptional start sites (TSSs), and the like.

Immunotherapy: As used herein, “immunotherapy” refers to treatment with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-40, OX40, B7.1, B7He, LAG3, CD137, KIR, CCR5, CD27, or CD40. Other exemplary agents include proinflammatory cytokines, such as IL-1β, IL-6, and TNF-α. Other exemplary agents are T-cells activated against a tumor, such as T-cells activated by expressing a chimeric antigen targeting a tumor antigen recognized by the T-cell.

Inactive: As used herein, “inactive” in the context of cfDNA fragments or molecules refers to molecules that originate from diseased cells (e.g., tumor cells). In certain embodiments, for example, a given “inactive” genomic region is a CTCF binding region that is not bound by a CTCF transcription factor.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Minor Allele Frequency: As used herein, “minor allele frequency” or “MAF” refers to the frequency at which minor alleles (e.g., not the most common allele) occur in a given population of nucleic acids, such as a sample obtained from a subject. In other words, “minor allele frequency” means the frequency of an allele observed at a given locus in a given sample that is not the most prevalent allele observed at that locus in that sample.

Mixture Model: As used herein, “mixture model” means a probabilistic model for representing the presence of subpopulations within an overall population, without requiring that an observed data set identifies the subpopulation to which an individual observation belongs.

MutantAllele Fraction: As used herein, “mutant allele fraction,” “mutation dose,” or “MAF” refers to the fraction of nucleic acid molecules harboring an allelic alteration or mutation at a given genomic position. MAF is generally expressed as a fraction or a percentage. For example, an MAF is typically less than about 0.5, 0.1, 0.05, or 0.01 (i.e., less than about 50%, 10%, 5%, or 1%) of all somatic variants or alleles present at a given locus.

Mutation: As used herein, “mutation” or “genetic aberration” refers to a variation from a known reference sequence and includes mutations such as, for example, single nucleotide variants (SNVs), copy number variants or variations (CNVs)/aberrations, insertions or deletions (indels), gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome.

Neoplasm: As used herein, the terms “neoplasm” and “tumor” are used interchangeably. They refer to abnormal growth of cells in a subject. A neoplasm or tumor can be benign, potentially malignant, or malignant. A malignant tumor is a referred to as a cancer or a cancerous tumor.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500, about 100, about 50 or about 10 nucleotides in length), used to label nucleic acid molecules to distinguish nucleic acids from different samples (e.g., representing a sample index), or different nucleic acid molecules in the same sample (e.g., representing a molecular tag), of different types, or which have undergone different processing. Nucleic acid tags can be single stranded, double stranded or at least partially double stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or both ends of the other nucleic acids (e.g., sample nucleic acids to be amplified and/or sequenced). Nucleic acid tags can be decoded to reveal information such as the sample of origin, form or processing of a given nucleic acid. Nucleic acid tags can also be used to enable pooling and/or parallel processing of multiple samples comprising nucleic acids bearing different nucleic acid tags and/or sample indexes in which the nucleic acids are subsequently being deconvoluted by reading the nucleic acid tags. Nucleic acid tags can also be referred to as molecular identifiers or tags, sample identifiers, index tags, and/or barcodes. Additionally or alternatively, nucleic acid tags can be used to distinguish different molecules in the same sample. This includes, for example, uniquely tagging each different nucleic acid molecule in a given sample, or non-uniquely tagging such molecules. In the case of non-unique tagging applications, a limited number of tags may be used to tag each nucleic acid molecule such that different molecules can be distinguished based on, for example, start/stop positions where they map to a selected reference genome in combination with at least one nucleic acid tag. Typically, a sufficient number of different nucleic acid tags are used such that there is a low probability (e.g., less than about a 10%, less than about a 5%, less than about a 1%, or less than about a 0.1% chance) that any two molecules will have the same start/stop positions and also have the same nucleic acid tag. Some nucleic acid tags include multiple molecular identifiers to label samples, forms of nucleic acid molecules within a sample, and nucleic acid molecules within a form having the same start and stop positions. Such nucleic acid tags can be referenced using the exemplary form “A1i” in which the uppercase letter indicates a sample type, the Arabic numeral indicates a form of molecule within a sample, and the lowercase Roman numeral indicates a molecule within a form.

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Reference Sequence: As used herein, “reference sequence” or “reference genome” refers to a known sequence used for purposes of comparison with experimentally determined sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” means anything capable of being analyzed by the methods and/or systems disclosed herein.

Sensitivity: As used herein, “sensitivity” in the context of a given assay or method refers to the ability of the assay or method to detect and distinguish between targeted (e.g., cfDNA fragments originating from tumor cells) and non-targeted (e.g., cfDNA fragments originating from non-tumor cells) analytes.

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and identity of monomer units (e.g., nucleotides, etc.) in that polymer.

Somatic Mutation: As used herein, “somatic mutation” means a mutation in the genome that occurs after conception. Somatic mutations can occur in any cell of the body except germ cells and accordingly, are not passed on to progeny.

Specificity: As used herein, “specificity” in the context of a diagnostic analysis or assay refers to the extent to which the analysis or assay detects an intended target analyte to the exclusion of other components of a given sample.

Statistical Transformation: As used herein, “statistical transformation” or “data transformation” in the context of data refers to a process transforms the data, generally by summarizing the information. In some implementations, statistical transformation involves normalizing a given data set.

Substantial Match: As used herein, “substantial match” means that at least a first value or element is at least approximately equal to at least a second value or element. In certain embodiments, for example, the cellular origin of at least the subset of the DNA molecules from a cfDNA sample is determined when there is at least a substantial or approximate match between a test sample distribution of cfDNA fragment properties and a reference sample distribution of cfDNA fragment properties.

Subject: As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Threshold: As used herein, “threshold” refers to a separately determined value used to characterize or classify experimentally determined values.

Tumor Fraction: As used herein, “tumor fraction” refers to the estimate of the fraction of nucleic acid molecules derived from tumor in a given sample. For example, the tumor fraction of a sample can be a measure derived from the maximum minor allele frequency (max MAF) of the sample or coverage of the sample, or length, epigenetic state, or other properties of the cfDNA fragments in the sample or any other selected feature of the sample. The term “max MAF” refers to the maximum or largest MAF of all somatic variants present in a given sample. In some embodiments, the tumor fraction of a sample is equal to the max MAF of the sample.

### DETAILED DESCRIPTION

### Introduction

The fragmentation pattern of cfDNA molecules in plasma or other sample types carries information about chromatin organization of the cells or tissues from which the cfDNA molecules originate. For example, DNA released into the bloodstream tends to be fragmented or cleaved around nucleosomes and/or other DNA bound proteins in the cells or tissues of origin. Nucleosome positioning and the location of DNA binding proteins is highly tissue specific and thus can be used to amplify signal coming from, for example, tumor as well as other cells or tissues contributing to a given sample's cfDNA fragment content.

The maintenance of DNA methylation or other epigenetic states is highly deregulated, for example, in tumor cells. Over the last few decades, for example extensive characterization of DNA methylation reprogramming has been documented across different cancer types, and numerous genomic regions of differential methylation between tumor tissues and normal tissues have been identified. In many cases, changes in DNA methylation or other epigenetic states are accompanied by changes in chromatin organization and nucleosome positioning within the same genomic region. Accordingly, combining these two sources of signal can significantly increase the ability to detect the presence of, for example, tumor cfDNA in plasma of early stage cancer patients as well as cfDNA originating from other cell and sample types.

This disclosure provides methods, computer readable media, and systems that are useful in determining the cellular origin of DNA molecules in cfDNA samples using properties of those cfDNA fragments, such fragment length, fragment midpoint relative the midpoint of a genomic region of the fragment, fragment epigenetic state, and/or other properties.

Methods and Related Aspects of Determining the Cellular Origin of CFDNA

This application discloses various methods related to determining whether cell-free DNA (cfDNA) samples comprise DNA molecules originating from given cell- or tissue-types. In some exemplary embodiments, the methods are used to determine whether a cfDNA sample includes DNA molecules originating from diseased cells (e.g., tumor cells, or the like), fetal cells, transplant donor cells, and/or the like. Frequently, these types of DNA molecules represent only a small fraction of all DNA molecules present in a given cfDNA sample, which generally includes a large background of DNA molecules originating from, for example, non-diseased, normal, or healthy cells (e.g., non-tumor cells, or the like), maternal cells, transplant recipient cells, and/or the like. Many pre-existing analytical techniques lack sufficient sensitivity to reliably detect and characterize DNA molecules present in such low numbers in cfDNA samples. The information obtained from the methods disclosed herein is typically used to diagnose whether a subject from whom the cfDNA sample was obtained has a given disease, disorder, or condition. In certain embodiments, the methods include administering therapy or otherwise treating the diagnosed disease, disorder, or condition in subjects. This application also discloses, for example, related methods of generating trained classifiers as well as methods of identifying biomarkers to use in determining the cellular origin of DNA molecules in cfDNA samples.

One exemplary embodiment uses both fragmentomics and epigenetic information. For each molecule, the fragmentomics information is represented by the genomic location of the molecule's end points. Whereas the epigenetic information captures epigenetic state of the molecule, such as DNA modification (5mC or 5hmC) state of CpG sites within the molecule or the identity of protein complexes bound to the molecule such as histone post-translation modifications, histone variants or specific transcription factors.

In certain implementations of this embodiment, a focused or targeted strategy is adopted where public and/or other data is used to identify a set of polymorphic loci. These are genomic regions that are expected to differ in chromatin organization between the normal/background state and disease/non-normal state. The term “chromatin organization” refers to the positioning of nucleosomes and other DNA binding protein complexes, and also to the epigenetic state which comprises, for example, DNA modifications and the identity of DNA bound protein complexes.

In some embodiments, the set of polymorphic loci or targeted panel uses in performing the methods, includes genomic regions, such as CTCF binding sites and transcription start sites (TSS). In certain embodiments, the targeted panel comprises genomic regions that are active in tissues contributing to the cfDNA of normal controls, but inactive in tissues contributing to the cfDNA of cancer patients, such as tissues comprising the tumor or the tumor micro-environment. For CTCF regions, the term “active” means that the genomic region is bound by CTCF and for TSS regions, the term “active” means that the corresponding transcript is actively transcribed in the tissue of origin. As described herein, the targeted panel optionally includes other genomic regions and/or genomic region categories.

In some embodiments, a probabilistic mixture model is used where DNA molecules originate from one of two sources a normal cell source (e.g., a non-tumor cell source, a maternal cell source, a transplant recipient cell source, etc.) or a targeted cell source (e.g., a tumor cell source, a fetal cell source, a transplant donor cell source, etc.). In these embodiments, the model parameters are typically estimated from a reference set of samples for which the fractional contribution of the targeted cell source is known. Once model parameters are estimated, the model is generally used to estimate the fractional contribution of targeted cell source θ for new samples using the following equations:

$${θ}_{ML} = arg {max}_{θ} Pr ( D \vert θ , Θ )$$

$$Pr ( D \vert θ , Θ ) = {Π}_{n} [ Pr ( {d}_{n} \vert {z}_{n} = targeted cell , Θ ) θ + Pr ( {d}_{n} \vert {z}_{n} = normal cell , Θ ) ( 1 - θ ) ]$$

where D is a collection of DNA molecules {d₁, d₂, . . . , d_{N}} from the new sample, dₙ is the set of observed variables associated with DNA molecule n, zₙ is the hidden/latent variable associated with DNA molecule n specifying its source either targeted cell or normal cell, θ is the set of the model parameters that are estimated from reference samples, and θ_{ML} is the Maximum Likelihood estimate of the fractional contribution of the targeted cell source.

An exemplary “instantiation” of the general model above describes the joint distribution of the following observed variables associated with each DNA molecule overlapping a genomic region: xₙ is the offset of the molecule midpoint with respect to the center of the region, yₙ is the length of the molecule, kₙ is the number of CpG di-nucleotides spanned by the molecule, and qₙ is the methyl binding domain (MBD) partition of the molecule. In these embodiments, dₙ=(xₙ,yₙ,kₙ,qₙ) and the joint distribution is given by:

Pr(xₙ,yₙ,kₙ,qₙ|Θ)=H(xₙ,yₙ)Pr(kₙ,qₙ|u)θ+F(xₙ,yₙ)Pr(kₙ,qₙ|v)(1−θ)

where Θ={H,F,u,v} are the parameters of the model estimated from a reference set of samples with known fractional contribution of targeted cell source. More specifically, H(x,y) is 2D density function specifying distribution of molecule midpoints and molecule lengths from targeted cell source, F(x,y) is a 2D density function specifying distribution of molecule midpoint offsets and molecule lengths from normal cell source, u is per CpG methylation rate in the targeted cell source, and v is per CpG methylation rate in the normal cell source.

In some embodiments, another “instantiation” of the general model above describes the joint distribution of the following observed variables associated with each DNA molecule overlapping a genomic region: xₙ is the offset of the molecule midpoint with respect to the center of the region and yₙ is the length of the molecule. In these embodiments, dₙ=(xₙ,yₙ) and the joint distribution is given by:

Pr(xₙ,yₙ|Θ)=H(xₙ,yₙ)θ+F(xₙ,yₙ)(1−θ)

where Θ={H, F} are the parameters of the model estimated from a reference set of samples with known fractional contribution of targeted cell source. More specifically, H(x,y) is 2D density function specifying distribution of molecule midpoints and molecule lengths from targeted cell source, and F(x,y) is a 2D density function specifying distribution of molecule midpoint offsets and molecule lengths from normal cell source.

In some embodiments, a reference set of normal cfDNA (i.e., samples with zero contribution from the targeted cell source (e.g., a tumor cell source, a fetal cell source, a transplant donor cell source, etc.)), the mean μ_{θ} and the standard deviation σ_{θ} of the distribution of θ values is used to compute and then to transform θ values into z-scores

$$z = \frac{θ - {μ}_{θ}}{{σ}_{θ}} .$$

In certain embodiments, a site-combined approach is used in which one θ estimate is derived from joint modeling of fragments overlapping multiple regions. In some of these embodiments, the corresponding z-score value z is used to decide whether the sample has non-zero contribution from the targeted cell source. In another exemplary approach, a separate θ estimate is derived from each region j. The corresponding z-score values zⱼ are used to decide whether the sample has non-zero contribution from the targeted cell source.

In certain embodiments, a statistical model is used to model the joint distribution of molecules in a cfDNA sample as a mixture of two components—one component representing normal and another component representing disease state. In these embodiments, each molecule is associated with a set of observed variables dₙ that represent observed fragmentomics and epigenetic information and a latent/hidden variable zₙ that represents the cell or tissue of origin normal or tumor. In these embodiments, the objective is to estimate the fraction of molecules originating from tumor, i.e., θ=Pr(zₙ=tumor).

In certain embodiments, the methods use the following observed variables dₙ=(xₙ,yₙ,kₙ,qₙ), where n is a given cfDNA fragment or molecule, xₙ is the offset of the midpoint with respect to the center or midpoint of the genomic region, yₙ is the molecule length, kₙ is the number of CpG sites, and qₙ is the methyl binding domain (MBD) partition. In other embodiments, the set of observed variables can be expanded to include other epigenetic information or new epigenetic information as it becomes available and/or molecule sequence information. Accordingly, the observed variables are in the general form dₙ.

In some embodiments, the methods use a maximum likelihood estimate of θ, i.e., an estimate that maximizes likelihood of the data:

$${θ}_{ML} = arg {max}_{θ} Pr ( D \vert θ , Θ )$$

$$Pr ( D \vert θ , Θ ) = {Π}_{n} [ Pr ( {d}_{n} \vert {z}_{n} = tumor , Θ ) θ + Pr ( {d}_{n} \vert {z}_{n} = normal , Θ ) ( 1 - θ ) ]$$

where Pr is probability, θ is the fraction of cfDNA fragments originating from an inactive or tumor source, ML is the maximum likelihood, D is a collection of DNA molecules {d₁, d₂, . . . , d_{N}} from the new sample, n is a given cfDNA fragment or molecules, and θ is the set of additional model parameters that are either estimated from control genomic regions on the targeted panel or from the reference set of normal and late stage tumor cfDNA samples. This estimate can also be generalized to evaluate other cfDNA targets, such as fetal/maternal cfDNA, transplant donor/transplant recipient cfDNA, or the like.

In some embodiments, each genomic region is treated separately. Thus, for each genomic region i and each sample j, an estimate θ_{i,j} is obtained. In certain of these embodiments, a set of reference normal samples is used to transform this estimate into a z-score z_{i,j}. Typically, the individual z-scores are then aggregated across genomic regions to obtain a tumor score sⱼ. Optionally, aggregation is obtained by computing the mean of theta z-scores z_{i,j}. In other exemplary embodiments, a machine learning algorithm is trained on z_{i,j} to predict whether a sample has a tumor component or not. Alternatively, data from the regions can be modeled together to obtain one estimate per sample θⱼ, which can be transformed into tumor score si.

Certain implementations of the methods disclosed herein are based on CTCF binding genomic regions. CTCF is transcription factor involved in many cellular processes including but not limited to transcription regulation and chromatin organization. Binding of CTCF is tissue specific, induces strong nucleosomal organization upstream and downstream of the binding site, and is DNA methylation sensitive. These perturbations of CTCF binding in tissues unique to plasma cfDNA of cancer patients are detectable from the fragmentomics and DNA methylation patterns around these sites using the methods disclosed in this application. However, applications of these methods are not limited to genomic regions, such as CTCF binding sites. Instead, the methods disclosed herein can be applied to essentially any other genomic region where epigenetic states (e.g., DNA methylation and/or the like) and nucleosome organization exhibit differences between or among cell/tissue types, including, for example, tissues uniquely contributing to plasma cfDNA of cancer patients. Examples of other genomic regions that can be used, include binding sites of transcription factors other than CTCF and transcription start sites (TSSs), among many others disclosed herein or otherwise known to those of ordinary skill in the art. Exemplary genomic regions are described further herein.

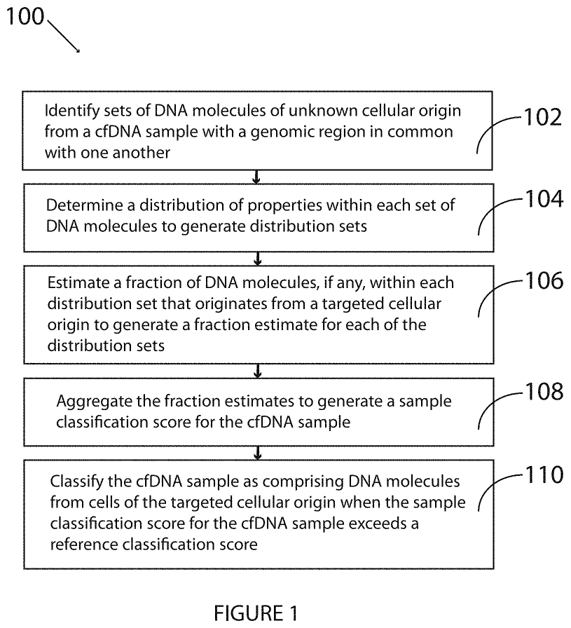

To further illustrate, FIG. 1 provides a flow chart that schematically depicts exemplary method steps of determining the cellular origin of DNA molecules from a cfDNA sample obtained from a subject according to some embodiments of the invention. As shown, method 100 includes identifying one or more sets of DNA molecules or fragments of unknown cellular origin from the cfDNA sample in step 102. Those sets each comprise one or more member DNA molecules that each comprise at least one genomic region in common with one another. The genomic regions are typically identified from sequence information obtained from the cfDNA sample by mapping that sequence information to one or more reference genome sequences. Typically, the genomic regions include regions of differential chromatin organization between at least two cell types. Exemplary genomic regions include transcriptional factor binding regions (e.g., CTCF binding regions or the like), distal regulatory elements (DREs), repetitive elements (e.g., microsatellites or the like), intron-exon junctions, transcriptional start sites (TSSs), and/or the like. Exemplary genomic regions are described further herein. Method 100 is generally at least partially implemented using a computer. Related systems comprising computers and computer readable media are described further herein.

Method 100 also includes determining a distribution of one or more properties among the member DNA molecules within each of the sets of DNA molecules from epigenetic information and/or the sequence information obtained from the cfDNA sample to generate one or more distribution sets in step 104. The properties typically include a length of a given DNA molecule, an offset of a midpoint of a given DNA molecule from a midpoint of the genomic region of the given DNA molecule, and/or an epigenetic status, pattern, or state exhibited by a given DNA molecule. Other properties are optionally used in lieu of, or in addition to, these properties. The length of a given DNA molecule can be determined, for example, once the endpoints (e.g., the respective 5′ and 3′ terminal nucleotides of a given strand from the DNA molecule) are identified by mapping the DNA molecule or fragment to a reference genome sequence. Other methods of measuring the length of a DNA molecule that are known to those of ordinary skill in the art are also optionally utilized. Similarly, the offset of the midpoint of a given DNA molecule from the midpoint of the genomic region of the given DNA molecule can also be determined from the identified length of the DNA molecule and the position of the genomic region in the DNA molecule, which can be obtained, for example, from the corresponding sequence information that is mapped to the reference genome sequence. The epigenetic status, pattern, or state exhibited by a given DNA molecule can be determined using essentially any technique known to those of ordinary skill in the art. When the epigenetic property being evaluated for a given DNA molecule or cfDNA fragment is, for example, DNA methylation (e.g., the CpG methylation pattern exhibited by the cfDNA fragment) that pattern is optionally determined using an agent that comprises a methyl-binding domain or methyl-CpG-binding domain (MBD) (e.g., a binding protein, an antibody, or any other agent capable of specifically binding to a modification of interest, such as 5-methylcytosine or the like) and/or a bisulfite sequencing technique, such as those suitable for use with Illumina® (Illumina, Inc., San Diego, Calif.) nucleic acid sequencing platforms. Other sequencing platforms or even other methods of DNA methylation analysis are also optionally utilized. Additional methods of DNA methylation analysis that are optionally adapted for use with the methods described herein are described, for example, in Kurdyukov et al., “DNA Methylation Analysis: Choosing the Right Method,” Biology (Basel), 5(1):3 (2016), which is incorporated by reference. DNA methylation patterns and other epigenetic information obtained from cfDNA fragments in performing these methods are described further herein. Additional details regarding the analysis of epigenetic modifications that are optionally adapted for use in performing the methods disclosed herein are described in, for example, WO 2018/119452, filed Dec. 22, 2017, which is incorporated by reference.

In addition, method 100 also includes estimating a fraction of member DNA molecules, if any, within each of the distribution sets that originate from a targeted cellular origin to generate a fraction estimate (e.g., an estimate of θ_{i,j}) for each of the distribution sets for the cfDNA sample in step 106. Essentially any cell- or tissue-type can be targeted using the methods described herein. In some embodiments, for example, the methods are used to determine whether a subject has a given disease, disorder, or disorder. In certain of these embodiments, the disease being assessed is cancer and accordingly, the targeted cellular origin of the DNA molecules or cfDNA fragments in a given sample are tumor cells. In other embodiments, the targeted cellular origin of the DNA molecules or cfDNA fragments in a given sample are, for example, fetal cells, organ transplant donor cells, infectious disease cells (e.g., bacterial cells or the like), or other cell types. Fraction estimates (e.g., an estimate of θ_{i,j}) for the distribution sets can also be determined using essentially any technique known to those of ordinary skill in the art. In some embodiments, for example, a mixture model or another approach to statistically transform the distribution set data is used to estimate or identify the fraction of cfDNA fragments in a given distribution set that originates from the targeted cell- or tissue-type. In some embodiments, a set of reference normal samples is used to transform individual fraction estimates into a z-score z_{i,j}. Suitable statistical transformation techniques, including mixture models, are described further herein or otherwise known to those of ordinary skill in the art. For example, additional details regarding statistical transformation techniques and modeling, including measures of statistical model accuracy, that are optionally adapted for using in performing the methods disclosed herein are provided in, for example, Bruce, Practical Statistics for Data Scientists: 50 Essential Concepts, 1^{st} Ed., O'Reilly Media (2017), Freedman et al., Statistics, 4^{th} Ed., W. W. Norton & Company (2007), James et al., An Introduction to Statistical Learning: with Applications in R, 1^{st} Ed., Springer (2013), and Hastie et al., The Elements of Statistical Learning: Data Mining, Inference, and Prediction, 2^{nd} Ed., Springer (2016), which are each incorporated by reference.

Method 100 also includes aggregating the fraction estimates (e.g., individual z-scores) for the cfDNA sample to generate a sample classification score (e.g., tumor score sⱼ) for the cfDNA sample in step 108. In addition, method 100 also includes classifying the cfDNA sample as comprising DNA molecules from cells of the targeted cellular origin (e.g., tumor cell origin) when the sample classification score (e.g., tumor score sⱼ) for the cfDNA sample exceeds a reference classification score to determine the cellular origin of at least the subset of DNA molecules or cfDNA fragments from the cfDNA sample obtained from the subject. In some embodiments, reference classification scores are obtained by applying method 100 to control samples obtained from subjects that are known to be healthy or in a normal state and/or from subjects that are known to have a given disease, disorder, or condition (e.g., a given type of cancer or the like). Although not shown in FIG. 1, once a cfDNA sample is determined to include cfDNA fragments that originate from, for example, diseased cells (e.g., tumor cells) and thus, diagnose the disease in the subject from whom the cfDNA sample was obtained, the methods also include administering one or more therapies to the subject based on the disease diagnosis to treat the disease in the subject in certain embodiments. Exemplary therapies are described further herein.

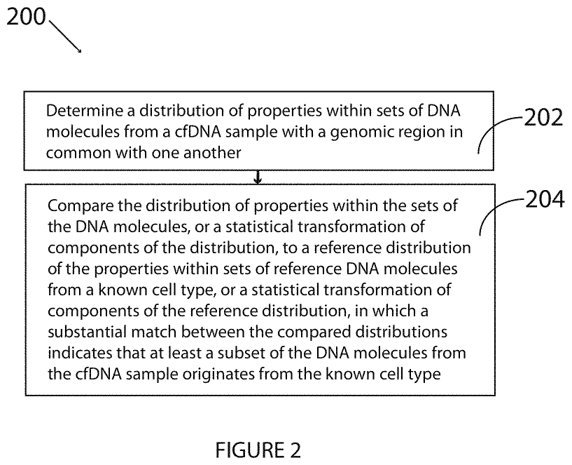

FIG. 2 provides a flow chart that schematically depicts exemplary method steps of determining the cellular origin of DNA molecules from a cfDNA sample obtained from a subject according to some embodiments of the invention. As shown, method 200 includes determining (typically using a computer) a distribution of one or more properties within sets of the DNA molecules from sequence and/or epigenetic information obtained from the DNA molecules or cfDNA fragments in step 202. Each set of the DNA molecules typically includes members that each comprise one or more genomic regions in common with one another. Typically, the genomic regions include regions of differential chromatin organization between at least two cell types. Exemplary genomic regions include transcriptional factor binding regions (e.g., CTCF binding regions or the like), distal regulatory elements (DREs), repetitive elements (e.g., microsatellites or the like), intron-exon junctions, transcriptional start sites (TSSs), and/or the like. The properties typically include a length of a given DNA molecule, an offset of a midpoint of a given DNA molecule from a midpoint of the genomic region of the given DNA molecule, and/or an epigenetic status, pattern, or state exhibited by a given DNA molecule.

Method 200 also includes comparing (typically using a computer) the distribution of the properties within the sets of the DNA molecules or cfDNA fragments, or a statistical transformation of one or more components of the distribution, to a reference distribution of the properties within sets of reference DNA molecules, or a statistical transformation of one or more components of the reference distribution in step 204. Suitable statistical transformation techniques, including mixture models, are described further herein or otherwise known to those of ordinary skill in the art. Each set of the reference DNA molecules comprises one or more members that each comprise one or more corresponding genomic regions in common with one another. In other words, each member DNA molecule in a given set of the reference DNA molecules has a given genomic region in common with one another and with a corresponding set of DNA molecules or cfDNA fragments from the cfDNA sample obtained from the subject in some embodiments. The reference DNA molecules typically originate from known cell types (e.g., normal cells, tumor cells, or the like). A substantial match between the distribution of the properties within the sets of the DNA molecules, or the statistical transformation of the components of the distribution, and the reference distribution of the properties within the sets of reference DNA molecules, or the statistical transformation of the components of the reference distribution, indicates that at least the subset of the DNA molecules from the cfDNA sample originates from the known cell types (e.g., normal cells, tumor cells, or the like) to thereby determine the cellular origin of at least the subset of the DNA molecules from the cfDNA sample obtained from the subject. When the subject is determined to have cfDNA fragments of a diseased cellular origin and hence, diagnose the disease in the subject, the methods further include administering one or more therapies to the subject based on the disease diagnosis to thereby treat the disease in the subject in some embodiments.

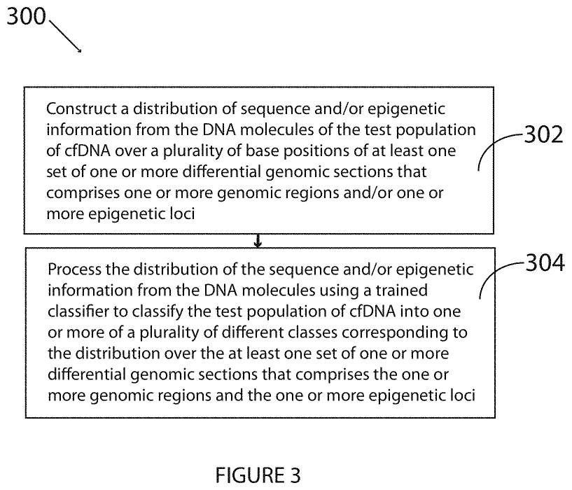

FIG. 3 provides a flow chart that schematically depicts exemplary method steps of classifying a test population of cfDNA from a subject at least partially using a computer according to some embodiments of the invention. As shown, method 300 includes constructing, by the computer, a distribution of sequence and/or epigenetic information from the DNA molecules or cfDNA fragments of the test population of cfDNA over a plurality of base positions of at least one set of one or more differential genomic sections that comprises one or more genomic regions and/or one or more epigenetic loci in step 302. Method 300 also includes processing, by the computer, the distribution of the sequence and/or epigenetic information from the DNA molecules using a trained classifier to classify the test population of cfDNA into one or more of a plurality of different classes corresponding to the distribution over the at least one set of one or more differential genomic sections that comprises the genomic regions and the epigenetic loci to thereby classify the test population of cfDNA from the subject in step 304.

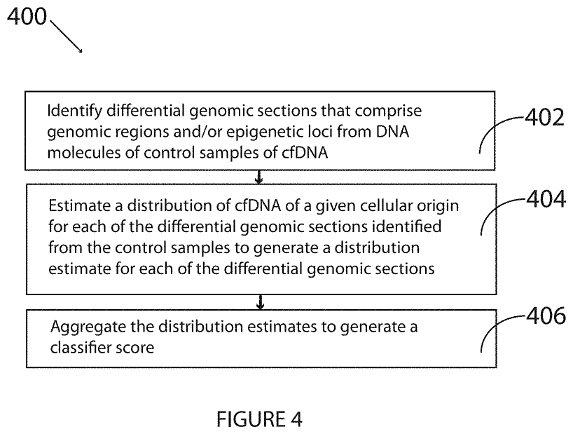

The present application also discloses various methods of generating trained classifiers of use, for example, in performing other methods described herein. To illustrate, FIG. 4 provides a flow chart that schematically depicts exemplary method steps of generating a trained classifier using a computer according to some embodiments of the invention. As shown, method 400 includes identifying, by the computer, at least one set of one or more differential genomic sections that comprises one or more genomic regions and/or one or more epigenetic loci from sequence and/or epigenetic information from DNA molecules or cfDNA fragments of a plurality of control samples of cfDNA in step 402. Method 400 also includes estimating, by the computer, a distribution of cfDNA of a given cellular origin (e.g., tumor origin, etc.) for each of the one or more differential genomic sections identified from the control samples to generate a distribution estimate for each of the one or more differential genomic sections in step 404. In addition, method 400 also includes aggregating, by the computer, the distribution estimates to generate a classifier score to thereby generating the trained classifier in step 406.

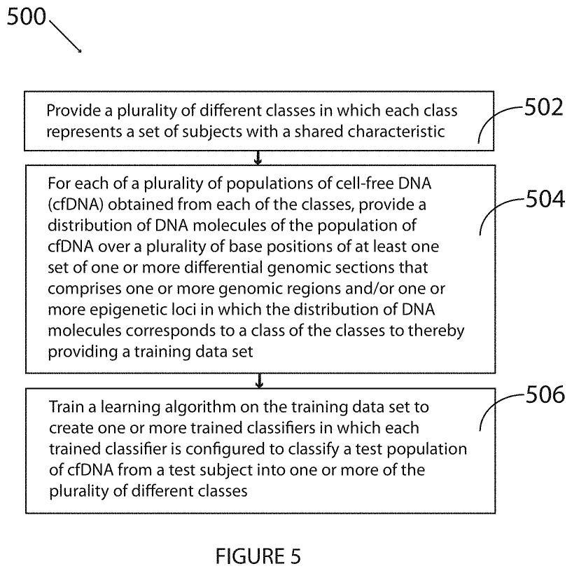

To further illustrate, FIG. 5 provides a flow chart that schematically depicts exemplary method steps of generating a trained classifier at least partially using a computer according to some embodiments of the invention. As shown, method 500 includes providing, by the computer, a plurality of different classes in which each class represents a set of subjects with a shared characteristic in step 502. Method 500 also includes for each of a plurality of populations of cfDNA obtained from each of the classes, providing, by the computer, a distribution of DNA molecules of the population of cfDNA over a plurality of base positions of at least one set of one or more differential genomic sections that comprises one or more genomic regions and/or one or more epigenetic loci in step 504. The distribution of DNA molecules corresponds to a class of the classes to thereby provide a training data set. In addition, method 500 also includes training a machine learning algorithm on the training data set to create one or more trained classifiers in which each trained classifier is configured to classify a test population of cfDNA from a test subject into one or more of the plurality of different classes in step 506.

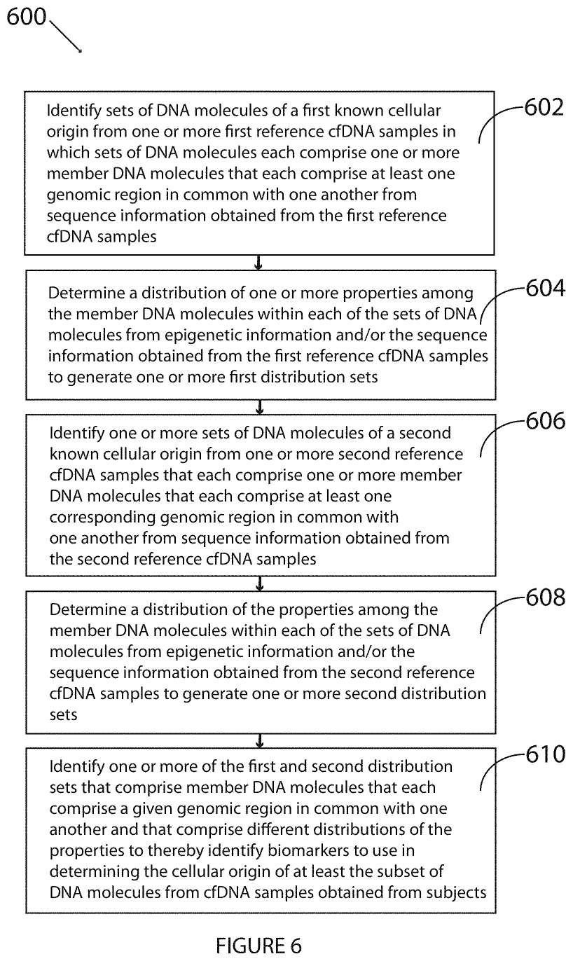

The present application also discloses various methods of identifying biomarkers of use, for example, in performing other methods described herein. To illustrate, FIG. 6 provides a flow chart that schematically depicts exemplary method steps of identifying biomarkers to use in determining a cellular origin of at least a subset of DNA molecules or cfDNA fragments from cfDNA samples obtained from subjects at least partially using a computer according to some embodiments of the invention. As shown, method 600 includes identifying one or more sets of DNA molecules of a first known cellular origin from one or more first reference cfDNA samples, which sets of DNA molecules each comprise one or more member DNA molecules that each comprise at least one genomic region in common with one another from sequence information obtained from the first reference cfDNA samples in step 602. Exemplary genomic regions include transcriptional factor binding regions (e.g., CTCF binding regions or the like), distal regulatory elements (DREs), repetitive elements (e.g., microsatellites or the like), intron-exon junctions, transcriptional start sites (TSSs), and/or the like. Method 600 also includes determining a distribution of one or more properties among the member DNA molecules within each of the sets of DNA molecules from epigenetic information and/or the sequence information obtained from the first reference cfDNA samples to generate one or more first distribution sets in step 604. The properties typically include a length of a given DNA molecule, an offset of a midpoint of a given DNA molecule from a midpoint of the genomic region of the given DNA molecule, and/or an epigenetic status, pattern, or state exhibited by a given DNA molecule.

As also shown, method 600 also includes identifying one or more sets of DNA molecules of a second known cellular origin from one or more second reference cfDNA samples that each comprise one or more member DNA molecules that each comprise at least one corresponding genomic region in common with one another from sequence information obtained from the second reference cfDNA samples in step 606. In other words, each member DNA molecule in a given set of DNA molecules from the second reference cfDNA samples has a given genomic region in common with one another and with a corresponding set of DNA molecules or cfDNA fragments from the first reference cfDNA samples in some embodiments. Method 600 also includes determining a distribution of the properties among the member DNA molecules within each of the sets of DNA molecules from epigenetic information and/or the sequence information obtained from the second reference cfDNA samples to generate one or more second distribution sets in step 608. In addition, method 600 also includes identifying one or more of the first and second distribution sets that comprise member DNA molecules that each comprise a given genomic region in common with one another and that comprise different distributions of the properties to thereby identify the biomarkers to use in determining the cellular origin of at least the subset of DNA molecules from cfDNA samples obtained from subjects in step 610.

In some embodiments, the methods include obtaining the cfDNA sample from a subject. Essentially any sample type is optionally utilized. In certain embodiments, for example, the cfDNA sample is tissue, blood, plasma, serum, sputum, urine, semen, vaginal fluid, feces, synovial fluid, spinal fluid, saliva, and/or the like. Additional exemplary sample types that are optionally utilized are described further herein. Typically, the subject is a mammalian subject (e.g., a human subject). Essentially any type of nucleic acid (e.g., DNA and/or RNA) can be evaluated according to the methods disclosed in this application. Some examples, include cell-free nucleic acids (e.g., cfDNA of tumor origin, fetal origin, maternal origin, and/or the like), cellular nucleic acids, including circulating tumor cells (e.g., obtained by lysing intact cells in a sample), circulating tumor nucleic acids, and the like.

The methods disclosed in this application generally include obtaining sequence information from nucleic acids in samples taken from subjects. In certain embodiments, the sequence information is obtained from targeted segments of the nucleic acids. Essentially any number of genomic regions are optionally targeted. The targeted segments can include at least 10, at least 50, at least 100, at least 500, at least 1000, at least 2000, at least 5000, at least 10,000, at least 20,000 or at least 50,000 (e.g., 25, 50, 75,100, 200, 300, 400, 500, 600, 700, 800, 900, 1,000, 2,000, 3,000, 4,000, 5,000, 6,000, 7,000, 8,000, 9,000, 10,000, 15,000, 25,000, 30,000, 35,000, 40,000, 45,000) different and/or overlapping genomic regions.

In these embodiments, the methods also typically include various sample or library preparation steps to prepare nucleic acids for sequencing. Many different sample preparation techniques are well-known to persons skilled in the art. Essentially any of those techniques are used, or adapted for use, in performing the methods described herein. For example, in addition to various purification steps to isolate nucleic acids from other components in a given sample, typical steps to prepare nucleic acids for sequencing include tagging nucleic acids with molecular identifiers or barcodes, adding adapters (e.g., which may include the barcodes), amplifying the nucleic acids one or more times, enriching for targeted segments of the nucleic acids (e.g., using various target capturing strategies, etc.), and/or the like. Exemplary library preparation processes are described further herein. Additional details regarding nucleic acid sample/library preparation are also described in, for example, van Dijk et al., Library preparation methods for next-generation sequencing: Tone down the bias, Experimental Cell Research, 322(1):12-20 (2014), Micic (Ed.), Sample Preparation Techniques for Soil, Plant, and Animal Samples (Springer Protocols Handbooks), 1^{st} Ed., Humana Press (2016), and Chiu, Next-Generation Sequencing and Sequence Data Analysis, Bentham Science Publishers (2018), which are each incorporated by reference in their entirety.

The methods disclosed herein are typically used to diagnose the presence of a disease, disorder, or condition, particularly cancer, in a subject, to characterize such a disease, disorder, or condition (e.g., to stage a given cancer, to determine the heterogeneity of a cancer, and the like), to monitor response to treatment, to evaluate the potential risk of developing a given disease, disorder, or condition, and/or to assess the prognosis of the disease, disorder, or condition. The methods disclosed herein are also optionally used for characterizing a specific form of cancer. Since cancers are often heterogeneous in both composition and staging, the data generated using the methods disclosed herein may allow for the characterization of specific sub-types of cancer to thereby assist with diagnosis and treatment selection. This information may also provide a subject or healthcare practitioner with clues regarding the prognosis of a specific type of cancer, and enable a subject and/or healthcare practitioner to adapt treatment options in accordance with the progress of the disease. Some cancers become more aggressive and genetically unstable as they progress. Other tumors remain benign, inactive or dormant.

Genomic Regions

In some embodiments, the methods, and related systems and computer readable media implementations, disclosed herein include identifying sets of DNA molecules or cfDNA fragments from cfDNA samples in which each member cfDNA fragment of a given set comprises a genomic region in common with one another. Essentially any genomic region can be used as long as cfDNA fragments comprising a given genomic region exhibit different properties (e.g., cfDNA fragment lengths, offsets of cfDNA fragment midpoints relative to midpoints of genomic regions comprised by the cfDNA fragment, epigenetic states, and/or the like) between at least two cell or tissue types. In certain embodiments, for example, genomic regions include regions of differential chromatin organization between at least two cell or tissue types. More specifically, fragmentation patterns of DNA molecules in cfDNA samples carries information about the chromatin organization of the cells or tissues from which the cfDNA fragments originate. In particular, DNA fragments released to the bloodstream is often fragmented or cleaved around nucleosomes and/or other DNA bound proteins in the cells or tissues of origin. Further, nucleosome positioning and the location of DNA binding proteins is highly tissue specific and thus is used herein to amplify signal coming from the cells or tissues from which the cfDNA fragments originate (e.g., tumor cells as well as cells in the tumor microenvironment and cells involved in the immune response). In certain embodiments, genomic regions comprise transcriptional factor binding regions, distal regulatory elements (DREs), repetitive elements, intron-exon or exon-intron junctions (splice junctions), transcriptional start sites (TSSs), and/or the like.

A transcription factor (or sequence-specific DNA-binding factor) is a protein that regulates the rate of transcription of genetic information from DNA to messenger RNA, by binding to a specific DNA recognition sequence. Transcription factors are also oftentimes involved in other cellular processes beyond transcriptional regulation. There are thought to around 2600 transcription factors in the human genome. A transcription factor includes at least one DNA-binding domain (DBD), which binds to a specific recognition sequence of DNA adjacent to the gene that it regulates. Non-limiting examples of transcription factors include CCCTC-binding factor (CTCF or 11-zinc finger protein)(recognition sequence: 5′-CCGCGNGGNGGCAG-3′ (SEQ ID NO: 1)), SP1 (recognition sequence: 5′-GGGCGG-3′), C/EBP (recognition sequence: 5′-ATTGCGCAAT-3′ (SEQ ID NO: 2)), AP-1 (recognition sequence: 5′-TGA(G/C)TCA-3′), c-Myc (recognition sequence: 5′-CACGTG-3′), ATF/CREB (recognition sequence: 5′-TGACGTCA-3′), and Oct-1 (recognition sequence: 5′-ATGCAAAT-3′). The genomic regions used in the methods described herein optionally include one or more of these or any other transcription factor recognition sequences or binding sites. Additional details regarding transcription factors and related recognition sequences are described in, for example, Latchman, “Transcription factors: an overview,” The International Journal of Biochemistry & Cell Biology, 29(12):1305-12 (1997) and Ptashne et al., “Transcriptional activation by recruitment,” Nature, 386(6625):569-77, which are incorporated by reference.

To further illustrate, CTCF is a transcription factor (also known as transcriptional receptor CTCF, 11-zinc finger protein, or CCCTC-binding factor) involved in many cellular processes, including but not limited to, transcription regulation and chromatin organization. Binding of CTCF can be tissue specific and can induce strong nucleosomal organization upstream and downstream of the CTCF binding site. Therefore, perturbation of such nucleosomal organization due to contribution of tissues unique to, for example, plasma cfDNA of cancer patients may be detected and revealed by analyzing the cfDNA fragment (fragmentomics) pattern in and around these sites (CTCF binding regions). Additional details regarding inferring genomic regions, such as CTCF binding sites and related aspects that are adapted for use in performing the methods described herein are disclosed in U.S. Provisional Application No. 62/692,495, filed Jun. 29, 2018, which is incorporated by reference.

Distal regulatory elements (DREs) are involved in transcription regulation and include locus control regions, enhancers, insulators, and silencing elements. Binding sites related to DREs are optionally used as genomic regions in the methods described herein. Additional details regarding DREs are described in, for example, Heintzman et al., “Finding distal regulatory elements in the human genome,” Curr Opin Genet Dev, Dec; 19(6):541-549 (2009), which is incorporated by reference.

Repetitive elements are recurring patterns of nucleotides that are present in multiple copies throughout a given genome and/or a population of genomes. Non-limiting examples of repetitive elements, include microsatellites, terminal repeats, tandem repeats, minisatellites, satellite DNA, interspersed repeats, transposable elements (e.g., DNA transposons, retrotransposons (e.g., LTR-retrotransposons (HERVs) and LTR-retrotransposons (HERVs)), etc.), clustered regularly interspaced short palindromic repeats (CRISPR), direct repeats, inverted repeats, mirror repeats, and everted repeats. The genomic regions used in the methods described herein optionally include one or more repetitive elements. Additional details regarding repetitive elements are described in, for example, de Koning et al., “Repetitive elements may comprise over two-thirds of the human genome,” PLoS Genet 7.12 (2011), which is incorporated by reference.

Exon/intron or intron/exon junctions (splice junctions) typically include specific duplex sequence patterns in genomes and are involved in RNA splicing of mRNA. These sequences are optionally used as genomic regions in the methods described herein. Additional details regarding exon/intron or intron/exon junctions and related sequences are described in, for example, Mount, “A catalogue of splice junction sequences,” Nucleic Acids Research, 10(2):459-472 (1982), which is incorporated by reference.

A transcription start site (TSS) is the location where the first DNA nucleotide at the 5′-end of a given gene is transcribed into RNA. TSS sequences are optionally used as genomic regions in the methods described herein. Additional details regarding TSSs are described in, for example, Farman et al., “Nucleosomes positioning around transcriptional start site of tumor suppressor (Rbl2/p130) gene in breast cancer,” Molecular Biology Reports, 45(2):185-194 (2018), which is incorporated by reference.

Epigenetic Information

In some embodiments, the methods, and related system and computer readable media implementations, disclosed herein include determining the cellular origin of DNA molecules from cfDNA samples using properties of those DNA molecules, such as epigenetic patterns exhibited by those molecules or fragments. As described herein, epigenetic changes in genomic sections are often accompanied by changes in chromatin organization and nucleosome positioning within those genomic sections. Accordingly, the methods and related aspects of this disclosure combine these sources of signal to increase the ability to detect the presence of targeted cells (e.g., diseased cells, such as tumor cells or the like), fetal cells, transplant donor cells, and the like) in cfDNA samples.

Any epigenetic site or locus that exhibits differential modifications (e.g., a post-replication modification or the like) between at least two cell or tissue types can be used to perform the methods and related aspects of the present disclosure. Examples of such sites, include methylation sites, acetylation sites, ubiquitylation sites, phosphorylation sites, sumoylation sites, ribosylation sites, citrullination sites, histone post-translational modification sites, histone variant sites, and/or the like. Examples of post-replication modifications, include 5-methyl-cytosine, 5-hydroxymethyl-cytosine, 5-carboxyl-cytosine, and 5-formyl-cytosine, among many others. Additional details regarding epigenetic sites or loci are described in, for example, Jin et al., “DNA Methylation: Superior or Subordinate in the Epigenetic Hierarchy?,” Genes Cancer, 2(6):607-617 (2011), Javaid et al., “Acetylation-and Methylation-Related Epigenetic Proteins in the Context of Their Target,” Genes (Basel), 8(8):196 (2017), Cao et al., “Histone Ubiquitination and Deubiquitination in Transcription, DNA Damage Response, and Cancer,” Front Oncol, 2:26 (2012), Rossetto et al., “Histone phosphorylation: A chromatin modification involved in diverse nuclear event,” Epigenetics, 7(10):1098-1108 (2012), Vranych et al., “SUMOylation and deimination of proteins: two epigenetic modifications involved in Giardia encystation,” Biochim Biophys Acta, 1843(9):1805-17 (2014), Sadakierska-Chudy et al., “A Comprehensive View of the Epigenetic Landscape. Part II: Histone Post-translational Modification, Nucleosome Level, and Chromatin Regulation by ncRNAs,” Neurotox Res, 27:172-197 (2015), Fuhrmann et al., “Protein Arginine Methylation and Citrullination in Epigenetic Regulation,” ACS Chem Biol, 11(3):654-668 (2016), Fan et al., “Metabolic regulation of histone post-translational modifications,” ACS Chem Biol, 10(1):95-108 (2015), and Henikoff et al., “Histone Variants and Epigenetics,” Cold Spring Harb Perspect Biol, 7(1) (2015), which are each incorporated by reference.

Epigenetic information can be obtained from cfDNA fragments using any technique known to those of ordinary skill in the art. In some embodiments, for example, DNA molecules from a given cfDNA sample are physically fractionated (e.g., fractionating with methyl-binding domain protein (“MBD”)-beads to stratify the cfDNA fragments into various degrees of methylation or the like) to generate partitions. In these embodiments, differential molecular tags and NGS-enabling adapters are applied to each of the two or more partitions to generate molecular tagged partitions. In addition, these embodiments also include assaying the molecular tagged partitions on an NGS instrument to generate sequence data for deconvoluting the sample into molecules that were differentially partitioned to generate the epigenetic information. In some embodiments, bisulfite sequencing techniques are also used to generate epigenetic information from cfDNA samples. Additional details regarding the analysis of epigenetic modifications that are optionally adapted for use in performing the methods disclosed herein are described in, for example, WO 2018/119452, filed Dec. 22, 2017, which is incorporated by reference.

Samples

A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations).

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

Nucleic Acid Tags

In certain embodiments, tags providing molecular identifiers or barcodes are incorporated into or otherwise joined to adapters by chemical synthesis, ligation, or overlap extension PCR, among other methods. In some embodiments, the assignment of unique or non-unique identifiers, or molecular barcodes in reactions follows methods and utilizes systems described in, for example, US patent applications 20010053519, 20030152490, 20110160078, and U.S. Pat. Nos. 6,582,908, 7,537,898, and 9,598,731, which are each incorporated by reference.

Tags are linked to sample nucleic acids randomly or non-randomly. In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique and/or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some embodiments, identifiers are predetermined, random, or semi-random sequence oligonucleotides. In other embodiments, a plurality of barcodes may be used such that barcodes are not necessarily unique to one another in the plurality. In these embodiments, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

Nucleic Acid Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular tags and/or sample indexes/tags to a nucleic acid molecule using conventional nucleic acid amplification methods. The amplifications are typically conducted in one or more reaction mixtures. Molecular tags and sample indexes/tags are optionally introduced simultaneously, or in any sequential order. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

Nucleic Acid Enrichment

In some embodiments, sequences are enriched prior to sequencing the nucleic acids. Enrichment is optionally performed for specific target regions or nonspecifically (“target sequences”). In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

Nucleic Acid Sequencing

Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

Sequencing Panel

To improve the likelihood of detecting genomic regions of interest and optionally, tumor indicating mutations, the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions (e.g., CTCF binding regions, CTCF binding sites, marker CTCF binding regions, and/or marker CTCF binding sites), for example, to detect a single cancer, a set of cancers, or all cancers. Alternatively, DNA may be sequenced by whole genome sequencing (WGS) or other unbiased sequencing method without the use of a sequencing panel. Examples of suitable panel and targets for use in panels can be found in the epigenetic targets described in U.S. provisional patent application 62/799,637, filed Jan. 31, 2019, which is incorporated by reference in its entirety.

In some aspects, a panel that targets a plurality of different genes or genomic regions (e.g., transcriptional factor binding regions, distal regulatory elements (DREs), repetitive elements, intron-exon junctions, transcriptional start sites (TSSs), and/or the like) is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

Some examples of listings of genomic locations of interest may be found in Table 1 and Table 2. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or 97 of the genes of Table 1. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, or 70 of the SNVs of Table 1. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 1. In some embodiments, genomic locations used in the methods of the present disclosure comprise at least 1, at least 2, at least 3, at least 4, at least 5, or 6 of the fusions of Table 1. In some embodiments, genomic locations used in the methods of the present disclosure comprise at least a portion of at least 1, at least 2, or 3 of the indels of Table 1. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or 115 of the genes of Table 2. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, at least 70, or 73 of the SNVs of Table 2. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 2. In some embodiments, genomic locations used in the methods of the present disclosure comprise at least 1, at least 2, at least 3, at least 4, at least 5, or 6 of the fusions of Table 2. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 2. Each of these genomic locations of interest may be identified as a backbone region or hot-spot region for a given bait set panel. An example of a listing of hot-spot genomic locations of interest may be found in Table 3. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 3. Each hot-spot genomic location is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic location of interest may seek to capture.

TABLE 1

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | |
| RIT1 | ROS1 | SMAD4 | SMO | SRC | STK11 | | | | |
| TERT | TP53 | TSC1 | VHL | | | | | | |

TABLE 2

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | ATM |
| RIT1 | ROS1 | SMAD4 | SMO | MAPK1 | STK11 | | | | |
| TERT | TP53 | TSC1 | VHL | MAPK3 | MTOR | | | | |
| NTRK3 | | | | | | | | | APC |
| | | | | | | | | | ARID1A |
| | | | | | | | | | BRCA1 |
| | | | | | | | | | BRCA2 |
| | | | | | | | | | CDH1 |
| | | | | | | | | | CDKN2A |
| | | | | | | | | | GATA3 |
| | | | | | | | | | KIT |
| | | | | | | | | | MLH1 |
| | | | | | | | | | MTOR |
| | | | | | | | | | NF1 |
| | | | | | | | | | PDGFRA |
| | | | | | | | | | PTEN |
| | | | | | | | | | RB1 |
| | | | | | | | | | SMAD4 |
| | | | | | | | | | STK11 |
| | | | | | | | | | TP53 |
| | | | | | | | | | TSC1 |
| | | | | | | | | | VHL |

TABLE 3

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and |
| | | | | | | deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, |
| | | | | | | L536, Y537, |
| | | | | | | D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, |
| | | | | | | 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, |
| | | | | | | D816, L818, |
| | | | | | | D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5:1295228 |
| TP53 | chr17 | 7573916 | 7574043 | 127 | 11 | Q331, R337, |
| | | | | | | R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

In some embodiments, the one or more regions in the panel comprise one or more loci from one or a plurality of genes for detecting residual cancer after surgery. This detection can be earlier than is possible for existing methods of cancer detection. In some embodiments, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect cancer in high risk patients earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A databased may comprise information about regulatory elements or genomic regions in tumor samples. The information relating to the sequenced tumor samples may include the frequency various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer 1 have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the minor allele frequency for a given genetic variant. The minor allele frequency may refer to the frequency at which minor alleles (e.g., not the most common allele) occurs in a given population of nucleic acids, such as a sample. Genetic variants at a low minor allele frequency may have a relatively low frequency of presence in a sample. In some cases, the panel allows for detection of genetic variants at a minor allele frequency of at least 0.0001%, 0.001%, 0.005%, 0.01%, 0.05%, 0.1%, or 0.5%. The panel can allow for detection of genetic variants at a minor allele frequency of 0.001% or greater. The panel can allow for detection of genetic variants at a minor allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The locations comprising genomic regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy may be can be quantified using measures such as sensitivity and specificity, predictive values, likelihood ratios, the area under the ROC curve, Youden's index and/or diagnostic odds ratio.

Accuracy may presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

Cancer and Other Diseases

In certain embodiments, the methods and aspects disclosed herein are used to diagnose a given disease, disorder or condition in patients. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Customized Therapies and Related Administration

In some embodiments, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain embodiments, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Systems and Computer Readable Media

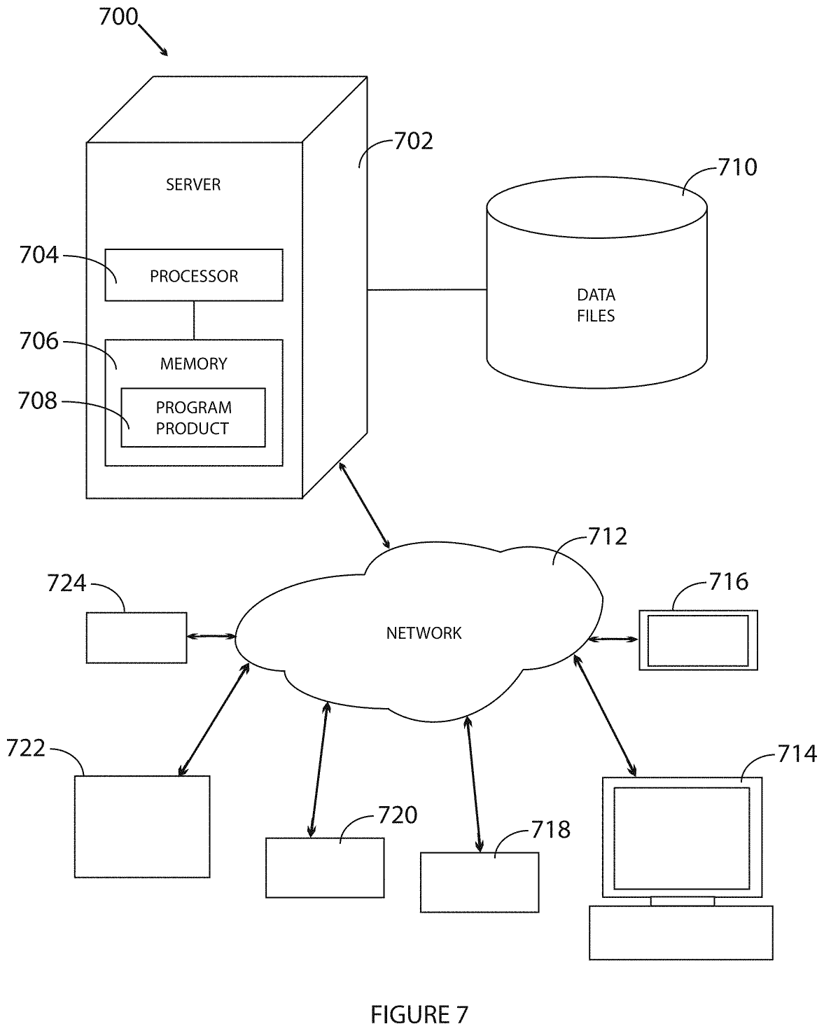

The present disclosure also provides various systems and computer program products or machine readable media. In some embodiments, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 7 provides a schematic diagram of an exemplary system suitable for use with implementing at least aspects of the methods disclosed in this application. As shown, system 700 includes at least one controller or computer, e.g., server 702 (e.g., a search engine server), which includes processor 704 and memory, storage device, or memory component 706, and one or more other communication devices 714 and 716 (e.g., client-side computer terminals, telephones, tablets, laptops, other mobile devices, etc.) positioned remote from and in communication with the remote server 702, through electronic communication network 712, such as the internet or other internetwork. Communication devices 714 and 716 typically include an electronic display (e.g., an internet enabled computer or the like) in communication with, e.g., server 702 computer over network 712 in which the electronic display comprises a user interface (e.g., a graphical user interface (GUI), a web-based user interface, and/or the like) for displaying results upon implementing the methods described herein. In certain embodiments, communication networks also encompass the physical transfer of data from one location to another, for example, using a hard drive, thumb drive, or other data storage mechanism. System 700 also includes program product 708 stored on a computer or machine readable medium, such as, for example, one or more of various types of memory, such as memory 706 of server 702, that is readable by the server 702, to facilitate, for example, a guided search application or other executable by one or more other communication devices, such as 714 (schematically shown as a desktop or personal computer) and 716 (schematically shown as a tablet computer). In some embodiments, system 700 optionally also includes at least one database server, such as, for example, server 710 associated with an online website having data stored thereon (e.g., classifier scores, control sample or comparator result data, indexed customized therapies, etc.) searchable either directly or through search engine server 702. System 700 optionally also includes one or more other servers positioned remotely from server 702, each of which are optionally associated with one or more database servers 710 located remotely or located local to each of the other servers. The other servers can beneficially provide service to geographically remote users and enhance geographically distributed operations.

As understood by those of ordinary skill in the art, memory 706 of the server 702 optionally includes volatile and/or nonvolatile memory including, for example, RAM, ROM, and magnetic or optical disks, among others. It is also understood by those of ordinary skill in the art that although illustrated as a single server, the illustrated configuration of server 702 is given only by way of example and that other types of servers or computers configured according to various other methodologies or architectures can also be used. Server 702 shown schematically in FIG. 7, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 700. As also understood by those of ordinary skill in the art, other user communication devices 714 and 716 in these embodiments, for example, can be a laptop, desktop, tablet, personal digital assistant (PDA), cell phone, server, or other types of computers. As known and understood by those of ordinary skill in the art, network 712 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 708 is optionally in the form of microcode, programs, cloud computing format, routines, and/or symbolic languages that provide one or more sets of ordered operations that control the functioning of the hardware and direct its operation. Program product 708, according to an exemplary embodiment, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 708 implementing the functionality or processes of various embodiments of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 708 is optionally copied from the computer-readable medium to a hard disk or a similar intermediate storage medium. When program product 708, or portions thereof, are to be run, it is optionally loaded from their distribution medium, their intermediate storage medium, or the like into the execution memory of one or more computers, configuring the computer(s) to act in accordance with the functionality or method of various embodiments. All such operations are well known to those of ordinary skill in the art of, for example, computer systems.

To further illustrate, in certain embodiments, this application provides systems that include one or more processors, and one or more memory components in communication with the processor. The memory component typically includes one or more instructions that, when executed, cause the processor to provide information that causes sequence information, epigenetic information, classifier scores, cfDNA property data, cfDNA fragment distribution set data, test results, control or comparator results, customized therapies, and/or the like to be displayed (e.g., via communication devices 714, 716, or the like) and/or receive information from other system components and/or from a system user (e.g., via communication devices 714, 716, or the like).

In some embodiments, program product 708 includes non-transitory computer-executable instructions which, when executed by electronic processor 704 perform at least: (i) receiving sequence and/or epigenetic information obtained from DNA molecules in a cfDNA sample obtained from a subject, (ii) identifying sets of DNA molecules of unknown cellular origin from the cfDNA sample that each comprise member DNA molecules that each comprise at least one genomic region in common with one another from the sequence information obtained from the cfDNA sample, (iii) determining a distribution of properties among the member DNA molecules within each of the sets of DNA molecules from epigenetic information and/or the sequence information obtained from the cfDNA sample to generate distribution sets, which properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule, (iv) estimating a fraction of member DNA molecules, if any, within each of the distribution sets that originate from a targeted cellular origin to generate a fraction estimate for each of the distribution sets for the cfDNA sample, (v) aggregating the fraction estimates for the cfDNA sample to generate a sample classification score for the cfDNA sample, and (vi) classifying the cfDNA sample as comprising DNA molecules from cells of the targeted cellular origin when the sample classification score for the cfDNA sample exceeds a reference classification score. Typically, program product 708 also includes non-transitory computer-executable instructions which, when executed by electronic processor 704 perform determining the properties among the member DNA molecules within each of the sets of DNA molecules from epigenetic information and/or the sequence information obtained from the cfDNA sample. Additional computer readable media embodiments are described herein.

System 700 also typically includes additional system components that are configured to perform various aspects of the methods described herein. In some of these embodiments, one or more of these additional system components are positioned remote from and in communication with the remote server 702 through electronic communication network 712, whereas in other embodiments, one or more of these additional system components are positioned local, and in communication with server 702 (i.e., in the absence of electronic communication network 712) or directly with, for example, desktop computer 714.

In some embodiments, for example, additional system components include sample preparation component 718 is operably connected (directly or indirectly (e.g., via electronic communication network 712)) to controller 702. Sample preparation component 718 is configured to prepare the nucleic acids in samples (e.g., prepare libraries of nucleic acids) to be amplified and/or sequenced by a nucleic acid amplification component (e.g., a thermal cycler, etc.) and/or a nucleic acid sequencer. In certain of these embodiments, sample preparation component 718 is configured to isolate nucleic acids from other components in a sample, to attach one or adapters comprising barcodes to nucleic acids as described herein, selectively enrich one or more regions from a genome or transcriptome prior to sequencing, and/or the like.

In certain embodiments, system 700 also includes nucleic acid amplification component 720 (e.g., a thermal cycler, etc.) operably connected (directly or indirectly (e.g., via electronic communication network 712)) to controller 702. Nucleic acid amplification component 720 is configured to amplify nucleic acids in samples from subjects. For example, nucleic acid amplification component 720 is optionally configured to amplify selectively enriched regions from a genome or transcriptome in the samples as described herein.

System 700 also typically includes at least one nucleic acid sequencer 722 operably connected (directly or indirectly (e.g., via electronic communication network 712)) to controller 702. Nucleic acid sequencer 722 is configured to provide the sequence information from nucleic acids (e.g., amplified nucleic acids) in samples from subjects. Essentially any type of nucleic acid sequencer can be adapted for use in these systems. For example, nucleic acid sequencer 722 is optionally configured to perform bisulfite sequencing, pyrosequencing, single-molecule sequencing, nanopore sequencing, semiconductor sequencing, sequencing-by-ligation, sequencing-by-hybridization, or other techniques on the nucleic acids to generate sequencing reads. Optionally, nucleic acid sequencer 722 is configured to group sequence reads into families of sequence reads, each family comprising sequence reads generated from a nucleic acid in a given sample. In some embodiments, nucleic acid sequencer 722 uses a clonal single molecule array derived from the sequencing library to generate the sequencing reads. In certain embodiments, nucleic acid sequencer 722 includes at least one chip having an array of microwells for sequencing a sequencing library to generate sequencing reads.

To facilitate complete or partial system automation, system 700 typically also includes material transfer component 724 operably connected (directly or indirectly (e.g., via electronic communication network 712)) to controller 702. Material transfer component 724 is configured to transfer one or more materials (e.g., nucleic acid samples, amplicons, reagents, and/or the like) to and/or from nucleic acid sequencer 722, sample preparation component 718, and nucleic acid amplification component 720.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### EXAMPLES

### Example 1: Generating a Representative Fragmentomics Profile of CTCF Binding

Under normal physiological conditions, cfDNA may be predominantly originating from tissues of hematopoietic lineage. Using public CTCF ChIP-Seq data for monocytes and neutrophils from ENCODE, a set of CTCF binding sites that are bound in both cell types were identified by taking an intersection of the top 10,000 strongest sites in both experiments, thereby obtaining a set of 6,902 sites. Next, a set of genomic regions comprising a local region of +/−1000 base pairs (bp) around the center of each of the set of sites was identified, and positions of fragments from our normal WGS data obtained from 19 normal (healthy) subjects were extracted and profiled at the set of genomic regions. Four different profiles were measured:

(1) Midpoint positions of short fragments, where short fragments have a length in a range of less than or equal to about 120 base pairs (bp).

(2) Midpoint positions of mono-nucleosomal fragments, where mono-nucleosomal fragments have a length in a range of greater than about 120 bp and less than or equal to about 240 bp.

(3) Start point positions of di-nucleosomal fragments, where di-nucleosomal fragments have a length in a range of greater than about 240 bp and less than or equal to about 400 bp.

(4) End (stop) point positions of di-nucleosomal fragments.

Profiles were generated for each of the set of genomic regions corresponding to CTCF binding sites as follows:

1. The number of events at a given genomic position (offset) was tallied (e.g., a number of fragments having a midpoint position, a start point position, or an end point position at the given offset).

2. Next, the signal was smoothed using a box filter with a width of 31 bp.

3. The signal was normalized to a 2001-bp length, e.g., to obtain an average of one event per genomic position (base-pair position). For example, normalization of a given 2001-bp genomic region can be performed by multiplying each value in the genomic region by 2001 and dividing by the sum of values across the genomic region.

4. Normalized profiles were truncated to [−400, 400] and concatenated, thereby resulting in a 4×801=3,204-dimensional representation for each CTCF binding site.

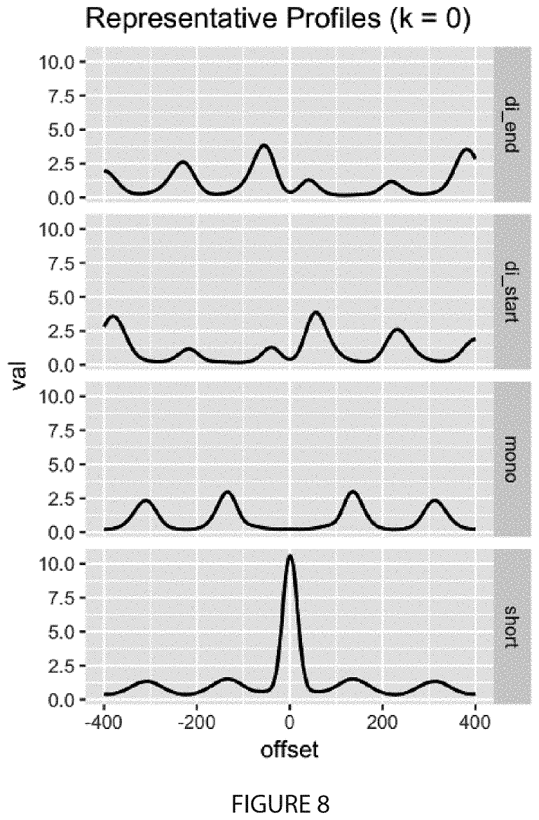

5. Cluster analysis was performed on the concatenated profile to identify a set of the most commonly occurring fragmentomics profile, thereby generating a representative fragmentomics profile. To illustrate, FIG. 8 is a plot showing an example of a representative CTCF profile.

### Example 2: Performing a Genome-Wide Scan for CTCF Binding Sites in Normal cfDNA

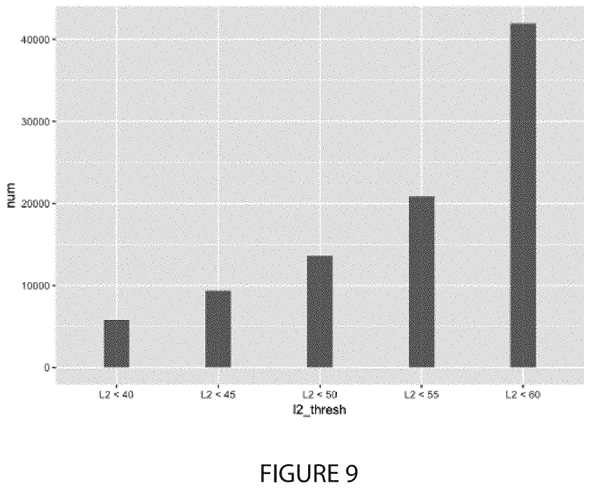

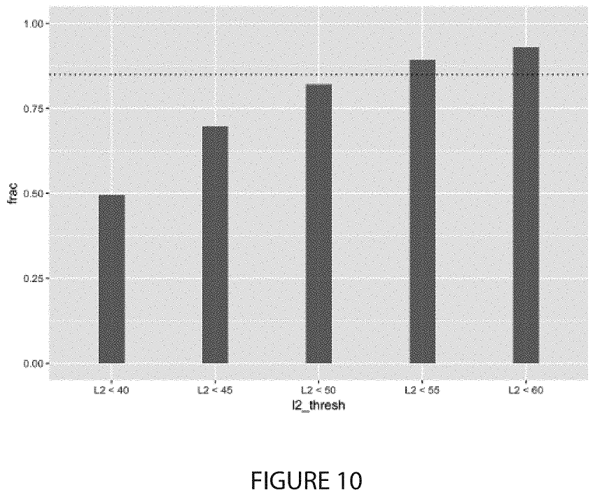

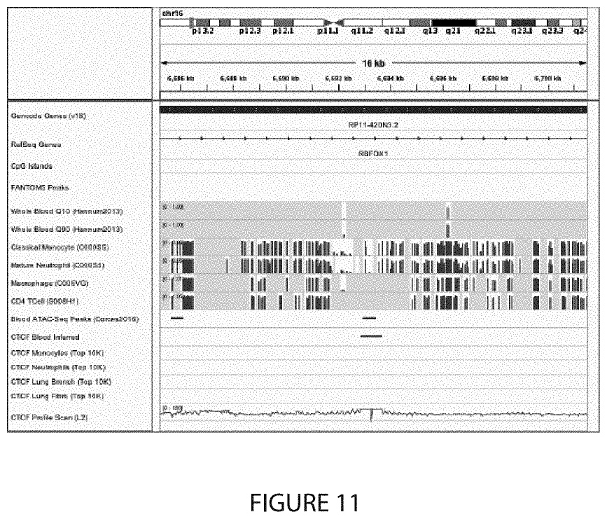

After generating a representative fragmentomics profile in Example 1, the profile was used to scan whole genome sequencing (WGS) data obtained from normal cfDNA samples (obtained from healthy subjects) for genomic loci having similar fragmentomics profiles. In particular, for each genomic position in the set of genomic regions, the fragmentomics profile around the genomic position was extracted according to the procedure described in Example 1. For each of the genomic positions, the Euclidean distance between the site profile at the genomic position and the representative profile was determined. Sites having a distance of less than 55 compared to the representative profile were identified as inferred CTCF binding sites, thereby obtaining a set of 20,869 such sites. For example, FIG. 9 shows a plot of a number of identified CTCF sites as a function of distance cut-off. As another example, FIG. 10 shows a plost of a fraction of known sites identified as a function of distance cut-off. FIG. 11 is a screenshot showing an example of an inferred CTCF site within an intronic region of the RBFOX1 gene. This site was not in the top 10,000 sites obtained from ENCODE CTCF ChIP-Seq data, thus demonstrating an example of the utility of the CTCF fragmentomics profiling approach. Additional details regarding inferring genomic regions, such as CTCF binding sites and related aspects that are adapted for use in performing the methods described herein are disclosed in U.S. Provisional Application No. 62/692,495, filed Jun. 29, 2018, which is incorporated by reference.

### Example 3: Selection of CTCF Binding Regions for Targeted Panel

The 20,869 CTCF inferred binding sites, referenced in Example 2, were overlapped with in-house plasma cfDNA methylation data and public tumor, adjacent normal and blood methylation data to select 39 CTCF Binding Regions for a targeted panel. Sites with low methylation levels around the center of the binding site in blood and elevated methylation levels in tumor samples were selected. These selection criteria guarantees that selected genomic regions are active (or bound by CTCF transcription factor) in tissues contributing to cfDNA in normal state and are inactive (or not bound by CTCF transcription factor) in tissues contributing to cfDNA in targeted cell state, which in this example are tumor tissues. In addition, focusing on regions of tumor specific DNA methylation allows for enrichment of tumor derived fragments in hyper and residual partitions. Finally, region inactivation is accompanied by both nucleosomal re-arrangement and gain of DNA methylation around the CTCF binding region and thus allows for powerful integration of fragmentomics and DNA methylation signals.

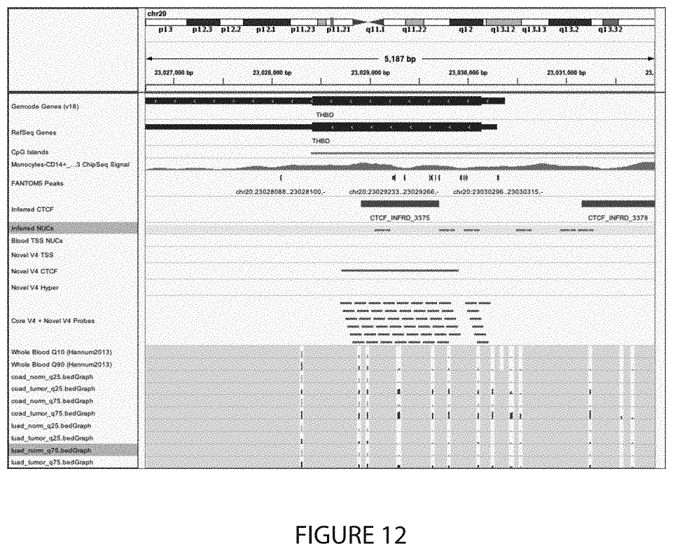

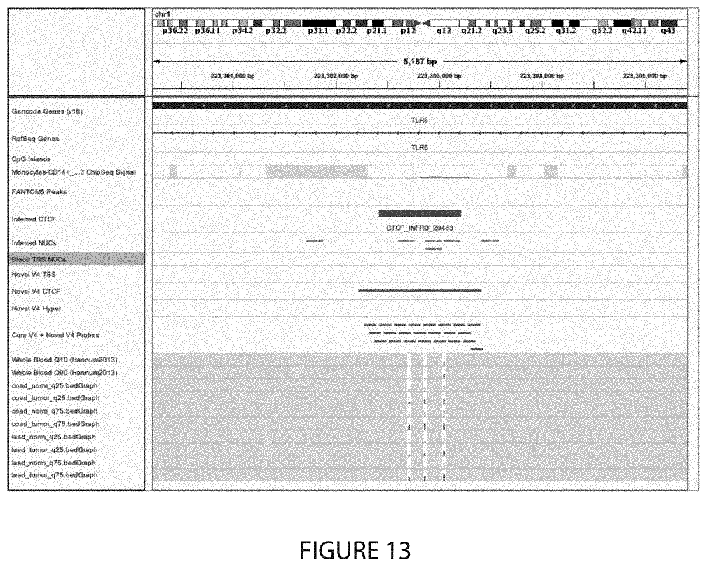

FIGS. 12 and 13 show genome browser screenshots of two selected regions: CTCF_INFRD_3375 and CTCF_INFRD_20483. The genome browser tracks include Gencode V18 and RefSeq gene annotations, inferred CTCF region boundaries, panel probes covering the selected region, 25th and 75th DNA methylation level quantiles derived from public blood methylation data, 25th and 75th DNA methylation level quantiles derived from TCGA COAD tumor and adjacent normal samples, 25th and 75th DNA methylation level quantiles derived from TCGA LUAD tumor and adjacent normal samples.

### Example 4: Estimation of Active and Inactive Density Functions

This example shows how to estimate F(x,y)=Pr(x,y|z=normal cell) and H(x,y)=Pr(x,y|z=targeted cell) density functions. These density functions specify joint distribution of fragment midpoint offsets and fragment lengths in normal/background state (active density function F(x,y)) and in targeted-cell state (inactive density function H(x,y)). One active and one inactive density function was estimated per CTCF binding region on the panel.

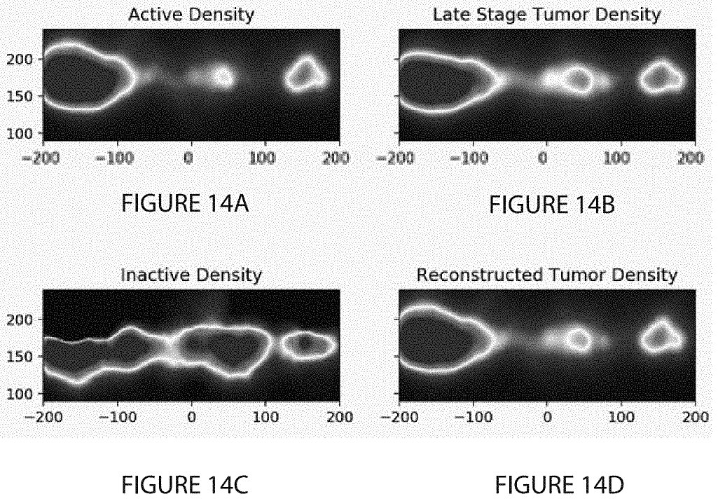

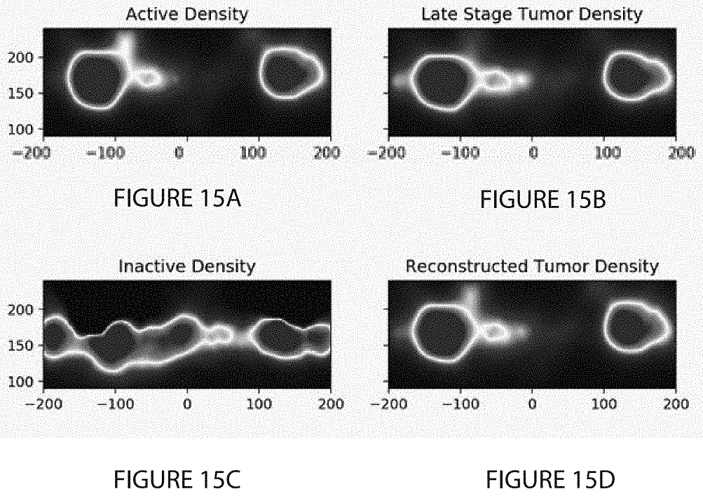

Given a CTCF binding region, a train set of Normal cfDNA samples (n=20) was used to estimate active density function F(x,y). DNA molecules overlapping the region were aggregated, and the number of molecules with given offsets and fragment lengths were counted. The resulting matrix of counts was transformed to a valid probability density function through smoothing and normalization. FIGS. 14A and 15A show active density function estimated for regions CTCF_INFRD_3375 and CTCF_INFRD_20483 respectively. The color gradient encodes the probability values across offset values ranging from −200 bp to 200 bp on the x-axis and fragment length values ranging from 90 bp to 240 bp on the y-axis; offset values correspond to the center of the inferred CTCF binding site.

Given a CTCF binding region, a train set of Late Stage High MAF (MAF 5%) tumor samples was used to estimate tumor density function X(x,y). The density estimation procedure is identical to one described above for the active density function F(x,y). FIGS. 14B and 15B show tumor density functions estimated for regions CTCF_INFRD_3375 and CTCF_INFRD_20483 respectively. The color gradient encodes the probability values across offset values ranging from −200 bp to 200 bp on the x-axis and fragment length values ranging from 90 bp to 240 bp on the y-axis; offset values correspond to the center of the inferred CTCF binding site.

The estimation of the inactive density function was modeled as a Maximum Likelihood Estimation Problem. More specifically, the estimated active density function F(x,y) and a fixed value of 0=0.1 were used to find H(x,y) that maximizes the probability of observed tumor data:

$$Pr ( D \vert F , H , θ ) = {Π}_{n} F ( {x}_{n} , {y}_{n} ) ( 1 - θ ) + H ( {x}_{n} , {y}_{n} ) θ$$

The formulation above translates into following optimization problem:

$${max}_{{H}_{i , j}} {\sum }_{i , j} {k}_{i , j} log [ ( 1 - θ ) {F}_{i , j} + θ {H}_{i , j} ) ]$$

$$\begin{matrix}s . t . {\sum }_{i , j} {H}_{i , j} = 1 & ( 1 ) \\ {H}_{i , j} \geq 0 & ( 2 )\end{matrix}$$

A solution to the optimization problem above that is not guaranteed to satisfy the inequality condition (2) is given by:

$$\begin{matrix}{\hat{H}}_{i , j} = \frac{1}{θ} \frac{{k}_{i , j}}{{Σ}_{i , j} {k}_{i , j}} - \frac{1 - θ}{θ} {F}_{i , j} \\ = \frac{1}{θ} {X}_{i , j} - \frac{1 - θ}{θ} {F}_{i , j}\end{matrix}$$

where X(i,j) is the tumor density function estimated above. The intermediate solution H is transformed into a valid density function through elimination of negative entries and normalization. The resulting inactive density functions for CTCF_INFRD_3375 and CTCF_INFRD_20483 regions are shown in FIGS. 14C and 15C and the reconstructed tumor density in FIGS. 14D and 15D respectively.

### Example 5: Formulation and Performance Evaluation of Fragmentomics Model

This example shows the performance of an exemplary model that utilizes only fragmentomics data to estimate targeted-cell fraction θ. Thus, per cfDNA fragment observed data dₙ consists of two variables: (i) xₙ is the offset of the fragment midpoint with respect to the center of the region and (ii) yₙ is the fragment length. Estimated active and inactive density functions (see Example 4) were used as well as the following model formulation:

$$Pr ( D \vert F , H , θ ) = {Π}_{n} H ( {x}_{n} , {y}_{n} ) θ + F ( {x}_{n} , {y}_{n} ) ( 1 - θ )$$

Given data from a cfDNA sample D={dₙ=(xₙ,yₙ)}, the formulation above and Maximum Likelihood were used to estimate per region θ values.

A pre-test set of Normal cfDNA samples (n=20) was used to compute the mean μ_{θ} and the standard deviation σ_{θ} of the distribution of θ values. The estimated μ_{θ} and σ_{θ} were used to transform θ values into z-scores

$$z = \frac{θ - {μ}_{θ}}{{σ}_{θ}} .$$

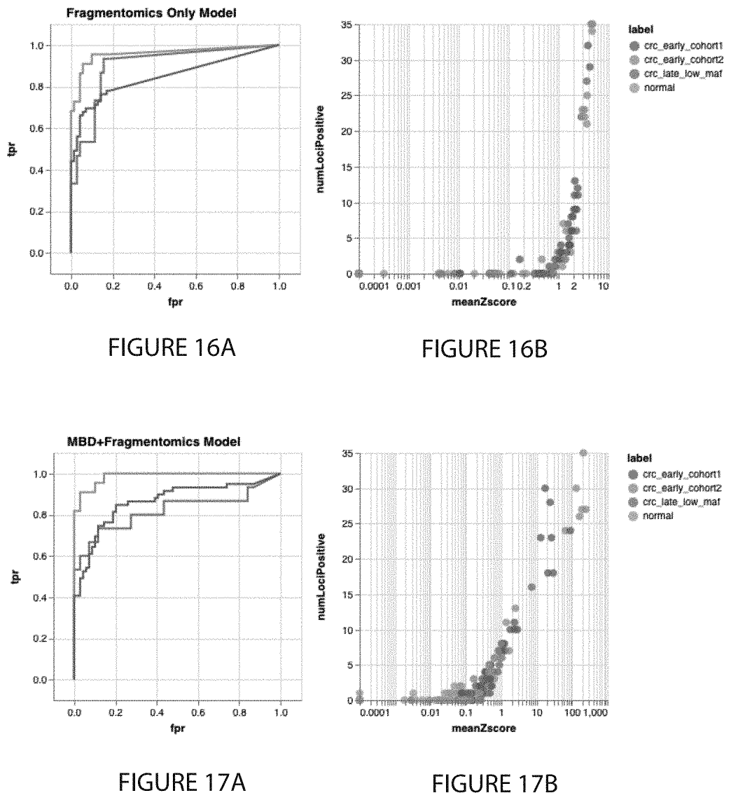

This example shows the performance of the model where per-region θ and z-score values were estimated as outlined above. The per-region z-score values zⱼ were aggregated by computing the mean of the z-score values and the number of regions with z-score values above 3.0. FIG. 16 summarizes the performance of the model on two different cohorts of Early Stage colorectal cancer (CRC) samples: crc_early_cohort1 (n=59) and crc_early_cohort2 (n=22). And also on a cohort of Late Stage Low-minor allele frequency (MAF) (MAF<2%) samples crc_late_low_maf (n=15). All evaluations were done against a cohort of age-matched normal cfDNA samples normal (n=70). FIG. 16A shows ROC curves and FIG. 16B shows the distribution of mean z-score values and the number of regions with z-score values above 3.0 across samples used in the evaluation. Samples and ROC curves are color-coded by the cohort.

Table 4 summarizes the cfDNA samples used to produce results shown in this and following example.

TABLE 4
Allocation of cfDNA samples for performance evaluation.

| | train/pre-test/ | number of |
|---|---|---|
| description | test | patients |
| early stage CRC tumor cohort 1 | test | 59 |
| early stage CRC tumor cohort 2 | test | 22 |
| normal | test | 70 |
| late stage CRC tumor low MAF | test | 15 |
| late stage CRC tumor high MAF | train | 20 |
| normal | train | 20 |
| normal | pre-test | 20 |

### Example 6: Formulation and Performance Evaluation of Fragmentomics an DNA Methylation Model

This example shows performance of an exemplary model that utilizes both fragmentomics and DNA methylation data to estimate targeted-cell fraction θ. Thus, per fragment observed data dₙ consists of four variables: (i) xₙ is the offset of the fragment midpoint with respect to the center of the region, (ii) yₙ is the fragment length, (iii) kₙ is the number of CpG sites overlapping the fragment, and (iv) qₙ is the methyl binding domain (MBD) partition of the fragment. Estimated active and inactive density functions were used (see Example 4) as well as per CpG methylation rate in normal state v, per CpG methylation rate in targeted-cell state u and the following model formulation:

$$Pr ( D \vert F , H , u , v , θ ) = {Π}_{n} H ( {x}_{n} , {y}_{n} ) Pr ( {k}_{n} , {q}_{n} \vert u ) θ + F ( {x}_{n} , {y}_{n} ) Pr ( {k}_{n} , {q}_{n} \vert v ) ( 1 - θ )$$

Given data from a cfDNA sample D={dₙ=(xₙ,yₙ,kₙ,qₙ))}, the formulation above and a Maximum Likelihood were used to estimate per region θ values.

A pre-test set of Normal cfDNA samples (n=20) was used to compute the mean μ_{θ} and the standard deviation σ_{θ} of the distribution of θ values. The estimated μ_{θ} and σ_{θ} were used to transform θ values into z-scores

$$z = \frac{θ - {μ}_{θ}}{{σ}_{θ}} .$$

This example shows the performance of the model where per-region θ and z-score values were estimated as outlined above. The per-region z-score values zⱼ were aggregated by computing the mean of the z-score values and the number of regions with z-score values above 3.0. FIG. 16 summarizes the performance of the model on two different cohorts of Early Stage CRC samples: crc_early_cohort1 (n=59) and crc_early_cohort2 (n=22). And also on a cohort of Late Stage Low-MAF (MAF<2%) samples crc_late_low_maf (n=15). All evaluations were done against a cohort of age-matched normal cfDNA samples normal (n=70). FIG. 17A shows ROC curves and FIG. 17B shows the distribution of mean z-score values and the number of regions with z-score values above 3.0 across samples used in the evaluation. Samples and ROC curves are color-coded by the cohort.

Table 4 summarizes the cfDNA samples used to produce results shown in this example.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise if different versions of a publication, website or the like are published at different times, the version most recently published at the effective filing date of the application is meant, unless otherwise indicated.

1. A method of determining a cellular origin of at least a subset of deoxyribonucleic acid (DNA) molecules from a cell-free DNA (cfDNA) sample obtained from a subject at least partially using a computer, the method comprising:
(a) identifying one or more sets of DNA molecules of unknown cellular origin from the cfDNA sample that each comprise one or more member DNA molecules that each comprise at least one genomic region in common with one another from sequence information obtained from the cfDNA sample;
(b) determining a distribution of one or more properties among the one or more member DNA molecules within each of the one or more sets of DNA molecules from epigenetic information and/or the sequence information obtained from the cfDNA sample to generate one or more distribution sets, which properties are selected from the group consisting of: a length of a given DNA molecule, an offset of a midpoint of a given DNA molecule from a midpoint of the at least one genomic region of the given DNA molecule, and an epigenetic status or pattern exhibited by a given DNA molecule;
(c) estimating a fraction of member DNA molecules, if any, within each of the one or more distribution sets that originate from a targeted cellular origin to generate a fraction estimate for each of the one or more distribution sets for the cfDNA sample;
(d) aggregating the fraction estimates for the cfDNA sample to generate a sample classification score for the cfDNA sample; and,
(e) classifying the cfDNA sample as comprising DNA molecules from cells of the targeted cellular origin when the sample classification score for the cfDNA sample exceeds a reference classification score, thereby determining the cellular origin of at least the subset of DNA molecules from the cfDNA sample obtained from the subject.

2-4. (canceled)

5. The method of claim 1, wherein the genomic regions comprise one or more regions of differential chromatin organization between at least two cell types.

6. The method of claim 1, wherein the genomic regions comprise one or more transcriptional factor binding regions, one or more distal regulatory elements (DREs), one or more repetitive elements, one or more intron-exon junctions, and/or one or more transcriptional start sites (TSSs).

7. The method of claim 6, wherein the one or more transcriptional factor binding regions comprise one or more CTCF binding regions.

8. The method of claim 1, wherein the epigenetic loci comprise one or more methylation sites, one or more acetylation sites, one or more ubiquitylation sites, one or more phosphorylation sites, one or more sumoylation sites, one or more ribosylation sites, one or more citrullination sites, one or more histone post-translational modification sites, and/or one or more histone variant sites.

9. The method of claim 8, wherein the epigenetic information comprises a methylation status of the one or more methylation sites, an acetylation status the one or more acetylation sites, a ubiquitylation status of the one or more ubiquitylation sites, a phosphorylation status of the one or more phosphorylation sites, a sumoylation status of the one or more sumoylation sites, a ribosylation status of the one or more ribosylation sites, a citrullination status of the one or more citrullination sites, a histone post-translational modification status of the one or more histone post-translational modification sites, and/or a histone variant status of the one or more histone variant sites.

10. The method of claim 1, wherein the epigenetic pattern comprises one or more of: a methylation pattern, an acetylation pattern, a ubiquitylation pattern, a phosphorylation pattern, a sumoylation pattern, a ribosylation pattern, a citrullination pattern, a histone post-translational modification pattern, and/or a histone variant pattern.

11. The method of 10, wherein the methylation pattern comprises a 5-methylcytosine (5mC) pattern and/or a 5-hydroxymethylcytosine (5hmC) pattern.

12. The method of claim 1, wherein the cellular origin of the subset of DNA molecules or the targeted cellular origin comprises a tumor cell.

13. The method of claim 1, wherein the cellular origin of the subset of DNA molecules or the targeted cellular origin comprises a non-tumor cell.

14. The method of claim 1, wherein the cellular origin of the subset of DNA molecules or the targeted cellular origin comprises a fetal cell.

15. The method of claim 1, wherein the cellular origin of the subset of DNA molecules or the targeted cellular origin comprises a maternal cell.

16. The method of claim 1, wherein the cellular origin of the subset of DNA molecules or the targeted cellular origin comprises a cell from a transplant donor subject.

17. The method of claim 1, wherein the cellular origin of the subset of DNA molecules or the targeted cellular origin comprises a cell from a transplant recipient subject.

18. The method of claim 1, wherein the cellular origin of the subset of DNA molecules or the targeted cellular origin comprises a non-diseased cell.

19. The method of claim 1, wherein the cellular origin of the subset of DNA molecules comprises a diseased cell, thereby diagnosing a disease in the subject.

20. The method of claim 1, further comprising administering one or more therapies to the subject to treat the disease in the subject.

21-26. (canceled)

27. The method of claim 1, comprising estimating a maximum likelihood that a fraction of DNA molecules in a given distribution set originates from the targeted cellular origin, using the equations of:
$${θ}_{ML} = arg {max}_{θ} Pr ( D \vert θ , Θ )$$
$$Pr ( D \vert θ , Θ ) = {Π}_{n} [ Pr ( {d}_{n} \vert {z}_{n} = targeted cell , Θ ) θ + Pr ( {d}_{n} \vert {z}_{n} = normal cell , Θ ) ( 1 - θ ) ]$$
where Pr is probability, θ is the fraction of DNA molecules in the given distribution set that originate from the targeted cellular origin, ML is the maximum likelihood, D is a collection of DNA molecules {d₁, d₂, . . . , d_{N}} from a test sample, n is a given DNA molecule in the given distribution set, dₙ is a set of observed variables that represent observed fragmentomics and epigenetic information, zₙ is a latent/hidden variable that represents a targeted or normal cell of origin, and Θ is a set of parameters that are estimated from control genomic regions on a targeted panel or from a reference set of cfDNA samples with DNA molecules from normal cells and cfDNA samples with DNA molecules from targeted cells.

28. The method of claim 27, wherein dₙ=(xₙ,yₙ,kₙ,qₙ), where n is the given DNA molecule in the given distribution set, xₙ is an offset of a midpoint of the given DNA molecule from a center of the genomic region of that given DNA molecule, yₙ is a length of the given DNA molecule, kₙ is a number of CpG sites in the given DNA molecule, and qₙ is a methyl binding domain (MBD) partition of the given DNA molecule.

29-108. (canceled)


SEQUENCE LISTING (2 sequences)

SEQ ID NO: 1 - DNA, 14 bp, Unknown
  source: /note=“Description of Unknown CTCF or 11-zinc finger recognition sequence”
  modified_base (6)..(6): a, c, t, g, unknown or other
  modified_base (9)..(9): a, c, t, g, unknown or other
ccgcgnggnggcag

SEQ ID NO: 2 - DNA, 10 bp, Unknown
  source: /note=“Description of Unknown C/EBP recognition sequence”
attgcgcaat